(19) United States
(12) Patent Application Publication
(10) Pub. No.: US 2024/0145071 A1
(43) Pub. Date: May 2, 2024

(54) Systems, Methods, And Apparatus For Providing A Medical Product Assistant To Recognize, Identify, And/Or Support A Medical Product

(71) Applicant: Cilag GmbH International, Zug (CH)

(72) Inventors: Christopher John Hess, Blue Ash, OH (US); Richard Royle Simmons, Loveland, OH (US); Roberta Hindi, Blue Ash, OH (US); Fabio Vieira Terracini, Blue Ash, OH (US); Karen Morgan Hess, Blue Ash, OH (US)

(21) Appl. No.: 18/375,195

(22) Filed: Sep. 29, 2023

Related U.S. Application Data
(60) Provisional application No. 63/421,859, filed on Nov. 2, 2022.

(51) Int. Cl. G16H 40/20; A61B 90/00; A61B 90/90; G16H 30/40

(52) U.S. Cl. CPC G16H 40/20; A61B 90/361; A61B 90/90; G16H 30/40

(57) ABSTRACT

Systems, methods, and apparatus may be provided for providing a medical product assistant to identify a medical product. A first message may be received from a user interface. The first message may indicate a request from a user to identify an object. A portion of the object may be determined using the device's camera. A confidence level associated with a medical product may be generated. The generated confidence level may indicate confidence that the object is the medical product. The generated confidence level may be based on the portion of the object and a product detection model. The product detection model may have been trained using synthetic product detection data that may comprise at least a computer-generated image of the medical product. The object may be identified as the medical product when the confidence level satisfies a threshold.

### CROSS-REFERENCE TO RELATED APPLICATIONS

This application claims the benefit of Provisional U.S. patent application No. 63/421,859, filed Nov. 2, 2022, the disclosure of which is incorporated herein by reference in its entirety.

### BACKGROUND

Medical products play a vital role in treating and managing diseases and symptoms. Information regarding a product may be important for the safe and effective use of medicines. However, a user may have difficulty finding information about a product. Such difficulties may lead to medical errors. Reducing medical errors by providing information regarding a medical product may be helpful in ensuring that the medical products are safely used.

### SUMMARY

Systems, methods, and apparatus may provide a medical product assistant that may use artificial intelligence for recognizing, identifying, and/or supporting a medical product. A first message may be received from a user interface. The first message may indicate a request from a user to identify an object. A portion of the object may be determined using the device's camera. A confidence level associated with a medical product may be generated. The generated confidence level may indicate confidence that the object is the medical product. The generated confidence level may be based on the portion of the object and a product detection model. The product detection model may have been trained using synthetic product detection data that may comprise at least a computer-generated image of the medical product. The object may be identified as the medical product when the confidence level satisfies a threshold. When the object is identified, a second message may be sent to the user interface. The second message may indicate that the object is the medical product. The second message may include a product identifier for the medical product, and/or medical product data.

Systems, methods, and apparatus may provide a medical product assistant that may use artificial intelligence for recognizing, identifying, and/or supporting a medical product. A first message may be received from a user interface. The first message may indicate a request from a user to identify an object. A portion of the object may be determined using the device's camera. A first confidence level associated with a first medical product may be generated. The generated first confidence level may indicate confidence that the object is the first medical product. The generated first confidence level may be based on the portion of the object and a product detection model. The product detection model may have been trained using synthetic product detection data comprising at least a computer-generated image of the first medical product. A second confidence level for the second medical product may be generated. The generated second confidence level may indicate confidence that the object is the second medical product. The object may be identified as the first medical product when the first confidence level is greater than the second confidence level, and the first confidence level satisfies a threshold. When the object is identified, a second message may be sent to the user interface. The second message may indicate that the object is the first medical product, a product identifier for the first medical product, and medical product data for the first medical product.

Systems, methods, and apparatus may provide a medical product assistant that may use artificial intelligence for recognizing, identifying, and/or supporting a medical product. A first message may be received from a user interface. The first message may indicate a request from a user to identify an object. A portion of the object may be determined using the device's camera. A confidence level associated with a medical product package may be generated. The generated confidence level may indicate confidence that the object is the medical product package. The generated confidence level may be based on the portion of the object and a package detection model. The package detection model may have been trained using synthetic package detection data that may comprise a computer-generated image of a package for a medical product. The object may be identified as the medical product when the confidence level satisfies a threshold. When the object is identified, a second message may be sent to the user interface. The second message may indicate that the object is the medical product, may indicate a product identifier for the medical product, and may indicate medical product data.

### BRIEF DESCRIPTION OF THE DRAWINGS

FIG. 1 depicts an example functional block diagram of electrical components of an example smart device for providing a medical product assistant that may be used for recognizing, identifying, and/or supporting a medical product.

FIG. 2A depicts an example architecture diagram for an example system to support a smart device and/or a smartwatch.

FIG. 2B is a messaging flow diagram for the example system.

FIG. 3 depicts a block diagram of a system that may include one or more modules (e.g., software modules) for providing a medical product assistant that may be used for recognizing, identifying, and/or supporting a medical product.

FIG. 4 depicts a block diagram of a system for collecting data and/or training artificial intelligence (AI) to be used by a medical product assistant.

FIG. 5A illustrates an example supervised learning framework.

FIG. 5B illustrates an example unsupervised learning framework.

FIG. 6 depicts a block diagram of a system for providing a medical product assistant that may be able to respond to requests from one or more audiences and/or contexts.

FIG. 7 depicts a block diagram of a system for providing a medical product assistant for delivering a personalized customer experience.

FIG. 8 depicts a block diagram of a system for providing a medical product assistant for delivering a personalized customer experience.

FIG. 9 depicts a block diagram that a medical product assistant may use for responding to one or more voice activation commands (e.g., wake words).

FIG. 10 is an example of a label and/or code that may be used to initiate a medical product assistant.

FIG. 11 depicts an example of one or more user interfaces that a medical product assistant may use to recognize, identify, and/or support a medical product.

FIG. 12 depicts an example of one or more user interfaces that a medical product assistant may use to support a medical product.

FIG. 13 depicts an example of one or more user interfaces that a medical product assistant may use for recognizing and/or identifying a medical product.

FIG. 14 depicts an example of one or more user interfaces that a medical product assistant may use for recognizing and/or identifying a medical product using encoded information associated with the medical product.

FIG. 15 depicts an example of one or more user interfaces that may be used by a medical product assistant to provide marketing material associated with a medical product.

FIG. 16 depicts an example of one or more user interfaces that may be used by a medical product assistant to provide marketing material associated with a medical product.

### DETAILED DESCRIPTION

Features described herein may include providing a user-centric (e.g., customer-centric) application to identify a medical product, improve the use of the medical product, and treat a medical condition that may be associated with the medical product.

Systems, methods, and apparatus may provide a medical product assistant that may use artificial intelligence to recognize, identify, and/or support a medical product.

Embodiments described herein may provide a medical product assistant that may use artificial intelligence to provide users (e.g., customers, health care providers, patients, etc.) with the information they request regarding a medical product in an intuitive manner. As disclosed herein, the medical product may be a medical instrument, such as an endocutter, a surgical stapler, and the like. The medical product may be a medication, such as antibiotics, painkillers, and the like. The medical product may be a medical device, such as a hearing aid, a pacemaker, and the like.

Systems, methods, and apparatus may provide a medical product assistant that may use artificial intelligence to recognize, identify, and/or support a medical product. A first message may be received from a user interface. The first message may indicate a request from a user to identify an object. A portion of the object may be determined using the device's camera. A confidence level associated with a medical product may be generated. The generated confidence level may indicate confidence that the object is the medical product. The generated confidence level may be based on the portion of the object and a product detection model. The product detection model may have been trained using synthetic product detection data that may comprise at least a computer-generated image of the medical product. The object may be identified as the medical product when the confidence level satisfies a threshold. When the object is identified, a second message may be sent to the user interface. The second message may indicate that the object is the medical product. The second message also include a product identifier for the medical product, and/or medical product data.

Systems, methods, and apparatus may provide a medical product assistant that may use artificial intelligence to recognize, identify, and/or support a medical product. A first message may be received from a user interface. The first message may indicate a request from a user to identify an object. A portion of the object may be determined using the device's camera. A first confidence level associated with a first medical product may be generated. The generated first confidence level may indicate confidence that the object is the first medical product. The generated first confidence level may be based on the portion of the object and a product detection model. The product detection model may have been trained using synthetic product detection data that may comprise at least a computer-generated image of the first medical product. A second confidence level for a second medical product may be generated. The generated second confidence level may indicate confidence that the object is the second medical product. The object may be identified as the first medical product when the first confidence level is greater than the second confidence level, and the first confidence level satisfies a threshold. When the object is identified, a second message may be sent to the user interface. The second message may indicate that the object is the first medical product, a product identifier for the first medical product, and medical product data for the first medical product.

Systems, methods, and apparatus may provide a medical product assistant that may use artificial intelligence to recognize, identify, and/or support a medical product. A first message may be received from a user interface. The first message may indicate a request from a user to identify an object. A portion of the object may be determined using the device's camera. A confidence level associated with a medical product package may be generated. The generated confidence level may indicate confidence that the object is the medical product package. The generated confidence level may be based on the portion of the object and a package detection model. The package detection model may have been trained using synthetic package detection data that may comprise a computer-generated image of a package for a medical product. The object may be identified as the medical product when the confidence level satisfies a threshold. When the object is identified, a second message may be sent to the user interface. The second message may indicate that the object is the medical product, may indicate a product identifier for the medical product, and may indicate medical product data.

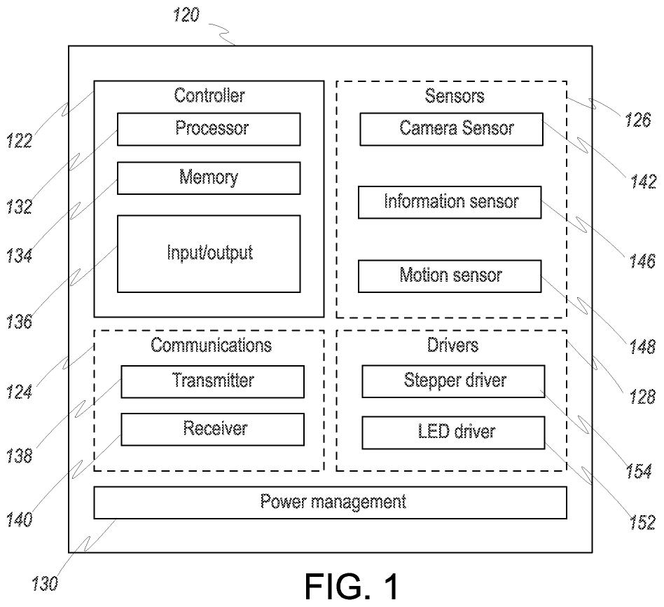

FIG. 1 depicts an example functional block diagram of electrical components of an example smart device that may be used to provide a medical product assistant. For example, FIG. 1 may depict an example functional block diagram of electrical components of an example smart device recognizing, identifying, and/or supporting a medical product.

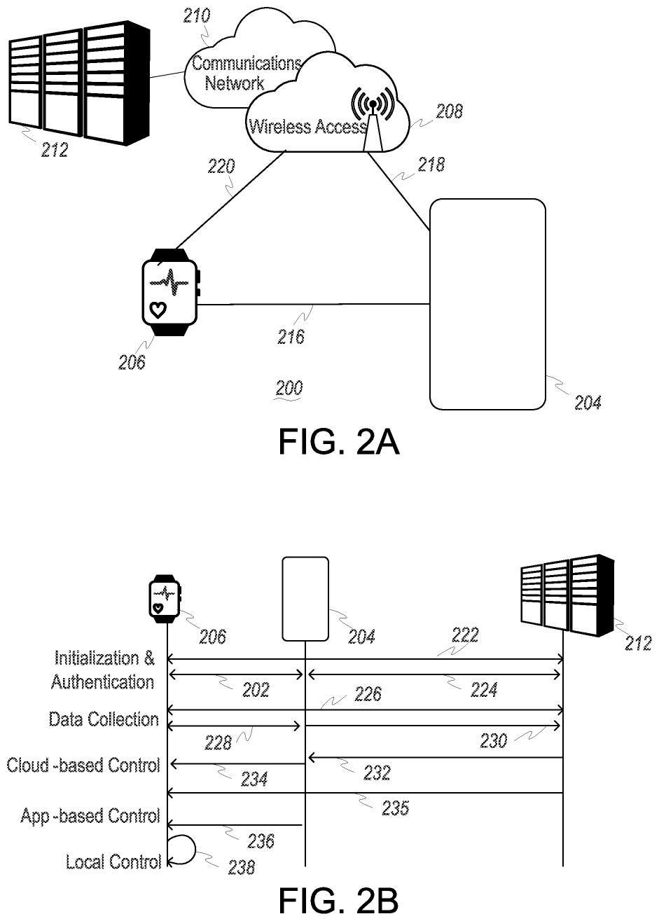

The smart device may be a smartphone, a tablet (e.g., an iPad), a smartwatch, a wearable device, a cellular phone, a computer, a server, and the like. Components 120 may be incorporated into the smart device, such as smartphone 204 and smartwatch 206 (shown in FIG. 2), and may be incorporated into a computing resource, such as 212 (also shown in FIG. 2). Referring again to FIG. 1, components 120 may integrate sensing, electromechanical driving, communications, and digital-processing functionality to the structure and operation of the device. In examples, components 120 may include a controller 122, communications interfaces 124, sensors 126, drivers 128 (which may include electrical and electromechanical drivers), and a power management subsystem 130.

Controller 122 may include, for example, a processor 132, a memory 134, and one or more input/output devices 136. Controller 122 may be any suitable microcontroller, microprocessor, field programmable gate array (FPGA), application-specific integrated circuit (ASIC), or the like that is suitable for receiving data, computing, storing, and driving output data and signals. Controller 122 may be a device suitable for an embedded application. For example, controller 122 may include a system on a chip (SOC).

Processor 132 may include one or more processing units. Processor 132 may be a processor of any suitable depth to perform the digital processing requirements disclosed herein. For example, processor 132 may include a 4-bit processor, a 16-bit processor, a 32-bit processor, a 64-bit processor, or the like. Processor 132 may include a graphic processing unit (GPU), an artificial intelligence (AI) processing unit, a machine learning processing unit, and other processors that may be appropriate for graphic processing, AI, and machine learning.

Memory 134 may include any component or collection of components suitable for storing data. For example, memory 134 may include volatile and/or nonvolatile memory. Memory 134 may include random-access memory (RAM), read-only memory (ROM), erasable programmable read-only memory (EPROM), (electrically erasable programmable read-only memory) EEPROM, flash memory, or the like.

Input/output devices 136 may include any apparatus suitable for receiving and sending information. This information may be in the form of digitally encoded data (from other digital components, for example) and analog data (from analog sensors, for example). Input/output devices 136 may include serial input/output ports, parallel input/output ports, universal asynchronous receiver transmitters (UARTs), discrete logic input/output pins, analog-to-digital converters, and digital-to-analog converters. Input/output devices 136 may include specific interfaces with computing peripherals and support circuitry, such as timers, event counters, pulse width modulation (P generators, watchdog circuits, clock generators, and the like. Input/output devices 136 may provide communication within and among the components 120, for example, communication between controller 122 and sensors 126, between controller 122 and drivers 128, between controller 122 and communications interfaces 124, and between controller 122 and the power management subsystem 130, and as a conduit for any other combination of components 120. Components 120 may support direct communication, for example, between sensor 126 and power management subsystem 130.

Communications interface 124 may include a transmitter 138 and/or a receiver 140. Communication interface 124 may include one or more transmitters 138 and/or receivers 140. Transmitter 138 and receiver 140 may include any electrical components suitable for communication to and/or from components 120. For example, transmitter 138 and receiver 140 may provide wireline communication and/or wireless communication to devices external to components 120 and/or external to the device within which components 120 are integrated.

Transmitter 138 and receiver 140 may enable wireline communication using any suitable communications protocol, for example, protocols suitable for embedded applications. For example, transmitter 138 and receiver 140 may be configured to enable universal serial bus (USB) communication, Ethernet local-area networking (LAN) communications, and the like.

Transmitter 138 and receiver 140 may enable wireless communications using any suitable communications protocol, for example, protocols suitable for embedded applications. For example, transmitter 138 and receiver 140 may be configured to enable a wireless personal area network (PAN) communications protocol, a wireless LAN communications protocol, a wide area network (WAN) communications protocol, and the like. Transmitter 138 and receiver 140 may be configured to communicate via Bluetooth, for example, with any supported or custom Bluetooth version and/or with any supported or customized protocol, including, for example, A/V Control Transport Protocol (AVCTP), A/V Distribution Transport (AVDTP), Bluetooth Network Encapsulation Protocol (BNEP), IrDA Interoperability (IrDA), Multi-Channel Adaptation Protocol (MCAP), and RF Communications Protocol (RFCOMM), and the like. In examples, transmitter 138 and receiver 140 may be configured to communicate via Bluetooth Low Energy (LE) and/or a Bluetooth Internet of Things (IoT) protocol. Transmitter 138 and receiver 140 may be configured to communicate via local mesh network protocols such as ZigBee, Z-Wave, Thread, and the like, for example. Such protocols may enable transmitter 138 and receiver 140 to communicate with nearby devices such as the user's cell phone and/or a user's smartwatch. And communication with a local networked device, such as a mobile phone, may enable further communication with other devices across a wide area network (WAN) to remote devices, on the Internet, on a corporate network, and the like.

Transmitter 138 and receiver 140 may be configured to communicate via LAN protocols such as 802.11 wireless protocols like Wi-Fi, including but not limited to communications in the 2.4 GHz, 5 GHz, and 60 GHz frequency bands. Such protocols may enable transmitter 138 and receiver 140 to communicate with a local network access point, such as a wireless router in a user's home or office. Communication with a local network access point may enable further communication with other devices present on the local network or across a WAN to remote devices, on the Internet, on a corporate network, and the like.

Transmitter 138 and receiver 140 may be configured to communicate via mobile wireless protocols such as a global system for mobile communications (GSM), 4G long-term evolution protocol (LTE), 5G, and 5G new radio (NR), and any variety of mobile Internet of things (IoT) protocols. Such protocols may enable transmitter 138 and receiver 140 to communicate more readily, for example, when a user is mobile, traveling away from their home or office, and without manual configuration.

Sensors 126 may include any device suitable for sensing an aspect of its environment, such as physical, chemical, mechanical, electrical, encoded information, and the like. Controller 122 may interact with one or more sensors 126. Sensors 126 may include, for example, camera sensor 142, information sensor 146, motion sensor 148, and the like. Although not shown, sensors 126 may include one or more biometric sensors such as a heart rate sensor, a blood oxygen sensor, a blood pressure sensor, a combination thereof, and the like.

Camera sensor 142 may include any sensor suitable for capturing and/or recording an image and/or video. Camera sensor 142 may be a charged-couple device (CCD), an active-pixel sensor, a complementary metal-oxide-semiconductor (CMOS) sensor, an N-type metal-oxide-semiconductor (NMOS) sensor, and the like. In examples, camera sensor 142 may be used to take an image or video of the medical product and/or medical product package. The image may be used to identify the medical product and/or information associated with the medical product. The text related to the image may be processed by a device comprising components 120.

In examples, camera sensor 142 may be used to take an image and/or video of information that may be encoded on a medical product and/or a medical product package. For example, the information may be encoded on the medical product and/or the medical product package using text, characters, numbers, a quick read (QR) code, a readable integrated circuit (e.g., a one-wire identification chip), a near-field communications (NFC) tag, in physical/mechanical keying, in a Subscriber Identification Module (SIM), or the like. For example, a user may use camera sensor 142 to take an image of a medical product, packaging for the medical product, a medical product label, and the like. The text associated with the image may be processed by a device comprising components 120.

Information sensor 146 may include any sensor suitable for reading stored information. In an embedded application with a physical platform, information may be encoded and stored on various media that may be incorporated into aspects of material design. For example, information about the medical product's authenticity and/or use may be incorporated into an aspect of the physical design of the medical product and/or the medical product packaging. In examples, the information may be encoded on the medical product and/or the medical product packaging using text, characters, numbers, a quick read (QR) code, a data matrix, a readable integrated circuit (e.g., a one-wire identification chip), a near-field communications (NFC) tag, radio frequency identification (RFID), in physical/mechanical keying, in a Subscriber Identification Module (SIM), and the like. The user may use the device to scan a QR code, and the device may communicate the information to controller 122 via communications interface 124. For example, information sensor 146 may be suitable for writing information back onto a medium associated with the readable code, such as a read/writable NFC tag.

Motion sensor 148 may include any sensor suitable for determining relative motion, acceleration, velocity, orientation, and the like. Motion sensor 148 may include a piezoelectric, piezoresistive, and/or capacitive component to convert physical motion into an electrical signal. For example, motion sensor 148 may include an accelerometer. Motion sensor 148 may include a microelectromechanical system (MEMS) device, such as a MEMS thermal accelerometer. Motion sensor 148 may be suitable for sensing a gesture made by the user intended to provide instruction to the medical product assistant. Motion sensor 148 may communicate this information via input/output devices 136 to processor 132 for processing.

A device comprising components 120 may include one or more drivers 128 to communicate feedback to a user and/or to drive a mechanical action. Drivers 128 may include a light-emitting diode (LED) driver 152, stepper driver 154, and the like. Drivers 128 may include haptic feedback drivers, audio output drivers, heating element drivers, and the like.

LED driver 152 may include any circuitry suitable for illuminating an LED. LED driver 152 may be controllable by the processor 132 via input/output devices 136. LED driver 152 may be used to indicate status information to a user. LED driver 152 may include a multicolor LED driver.

Stepper driver 154 may include any circuitry suitable for controlling a stepper motor. Stepper driver 154 may be controllable by processor 132 via input/output devices 136. Stepper driver 154 may be used to control a stepper motor associated with a medical device. For example, stepper driver 154 may be used to control a stepper motor of an insulin pump or a motor of a prosthetic arm.

Power management subsystem 130 may include circuitry suitable for managing and distributing power to the components 120 of a smart device. Power management subsystem 130 may include a battery, a battery charger, and a direct current (DC) power distribution system. Power management subsystem 130 may communicate with processor 132 via input/output devices 136, to provide information, such as battery charging status. Power management subsystem 130 may include a replaceable battery and/or a physical connector to enable external charging of the battery.

FIG. 2A depicts an example architecture diagram for an example system to support a device, such as a smart device. System 200 may include a smartphone 204 with a corresponding application (e.g., app), a smartwatch 206 with a corresponding app, a wireless access network 208, a communications network 210, and a computing resource 212.

The smart device may be a smartphone, a tablet (e.g., an iPad), a smartwatch, a wearable device, a cellular phone, a computer, a server, and the like. The smart device may be the smart device shown in FIG. 1 and may be smartwatch 206, smartphone 204, and/or computing resource 212 shown in FIGS. 2A-B.

Referring again to FIG. 2A, smartphone 204 may include an app. The app may be a medical product assistant for recognizing, identifying, and/or supporting a medical product. Smartphone 204 may provide passive tracking, active tracking, and/or location services. Smartphone 204 may collect data regarding the user, process data regarding the user, and/or share data regarding the user. For example, smartphone 204 may be able to use one of its sensors to collect information regarding a medical product and may be able to share that data with smartwatch 206 and/or computing resource 212. As another example, smartphone 204 may be able to determine that a user has used a medical product and may be able to share that data with smartwatch 206 and/or computing resource 212.

Smartwatch 206 may be able to provide information regarding a medical product to a user. Smartwatch 206 may provide biometric feedback and data such as heart rate and/or heart rate variability. Smartwatch 206 may perform activity tracking and may provide activity information. Smartwatch 206 may be used by a user to request information regarding a medical product. For example, a user may verbally ask the medical product assistant for information regarding a medical product.

Computing resources 212 may provide data storage and processing functionality. Computing resources 212 may receive and analyze medical product data and/or medical product usage data. Computing resources 212 may be or may include one or more servers. Computing resources 212 may include information regarding a user, such as purchasing information associated with the user, marketing information associated with the user, and user business information associated with the user. Computing resources 212 may include a purchasing history of one or more medical products that the user may use. Computer resource 212 may include a history of requests for medical information from one or more users. The medical product assistant may use the history of requests to anticipate the information a user may request.

The components of system 200 may communicate with each other over various communications protocols. Smartwatch 206 may communicate with the smartphone 204 over a link, such as wireless link 216, which may be a Bluetooth wireless link. Smartphone 204 may communicate with the wireless access network 208 over a link, such as wireless link 218. Smartwatch 206 may communicate with wireless access network 208 over a link, such as wireless link 220. Wireless link 218 and/or wireless link 220 may include any suitable wireless protocol, such as 802.11 wireless protocols like Wi-Fi, GSM, 4G LTE, 5G, and 5G NR, any mobile IoT protocols, and the like.

Communications network 210 may include a long-distance data network, such as a private corporate network, a virtual private network (VPN), a public commercial network, an interconnection of networks, such as the Internet, and the like. Communications network 210 may provide connectivity to computing resource 212.

Computing resource 212 may include any server resources suitable for remote processing and/or storing of information. For example, computing resource 212 may include a server, a cloud server, a data center, artificial intelligence, machine learning resources, models for artificial intelligence, models for machine learning, a virtual machine server, and the like. In examples, smartwatch 206 may communicate with computing resource 212 via wireless link 220, and smartwatch 206 may communicate with computing resource 212 via wireless link 218. Computing resource 212 may include any server resources suitable for remote processing and/or storing of information.

System 200 may assist the medical product assistant in recognizing, identifying, and/or supporting a medical product. For example, system 200 may address the request of health care providers (HCPs) by providing information (e.g., reliable and up-to-date information) in various formats. The medical product assistant may receive and respond to requests visually (e.g., text, images, gestures, etc.) and/or by voice. The medical product assistant may use voice activation and visual recognition to provide personalized access. The medical product assistant may provide information tailored to fit a user's specific requests (e.g., a healthcare professional). The medical product assistant may be used as a decision support tool that may provide suggestions (e.g., real-time suggestions) based on research, clinical evidence, and medical product information.

FIG. 2B is an example messaging flow diagram for system 200. System 200 may include communication and processing for functions such as initialization and authentication of smartphone 204 and/or a medication app; data collection from smartwatch 206 and/or smartphone 204; cloud base control, triggering, notification messaging, and the like; app-based control, messaging and notifications, and the like.

Initialization and authentication message 222 may be exchanged between one or more of smartwatch 206, smartphone 204, or computing resource 212. For example, a user may create a user account via the smartphone 204. The account information may be processed by computing resource 212. The user (e.g., a new user) may initialize smartwatch 206 and/or wish to authenticate smartwatch 206. The information may be communicated via messaging 202 to the smartphone 204 and then via initialization and authentication messaging 224 to computing resources 212. The information may be communicated via initialization and authentication messaging 222 to computing resources 212. Responsive information about user accounts, medication usage, medication consumption, medications associated with a user, and the like may be messaged back to smartwatch 206 and/or smartphone 204.

Data collection functionality may be provided and may include messaging 226 from the smartwatch 206, smartphone 204, and/or computing resource 212. This messaging may include information such as medical product information, medical product usage information, medical product sale information, medical product marketing information, activity information, heart rate, heart rate variability, medication consumption, medication information, electronic medication records, medical data regarding a patient, prescriptions, and the like. The data collection functionality may include aggregate messaging 228 from smartwatch 206 to smartphone 204. In examples, smartphone 204 may aggregate messaging 228, process it locally, and/or communicate it or related information to computing resources 212 via message 230.

System 200 enables cloud-based control functions, app-based control functions, and local control functions. For example, medical product information, medical product usage information, medical product sale information, medical product marketing information, medication data, medication consumption data, active ingredient data, statuses, and/or reporting may be provided from computing resources 212 to smartphone 204 via messaging 232, and if appropriate, from smartphone 204 to smartwatch 206 via message 234. Computing resource 212 may communicate directly to the smartwatch 206 using messaging 235.

In examples, medical product information may be retrieved by the medical product assistant and may be displayed on smartphone 204 and/or on smartwatch 206. The medical product assistant may be computing resources 212, smartphone 204, and/or smartwatch 206. The personalized medication data, statuses, and/or reporting may be communicated to smartwatch 206 via message 236.

In examples, smartwatch 206 may provide local control via its local processor. Internal system calls and/or local messaging are illustrated as a local loop 238. For example, smartwatch 206 may provide a medical product assistant for recognizing, identifying, and/or supporting a medical product.

Embodiments described herein may provide a medical product assistant that may use artificial intelligence to provide users (e.g., customers, health care providers, patients, etc.) with the information they request regarding a medical product intuitively. As disclosed herein, the medical product may be a medical instrument, such as an endocutter, a surgical stapler, and the like. The medical product may be a medication, such as antibiotics, painkillers, and the like. The medical product may be a medical device, such as a hearing aid, a pacemaker, and the like.

To address the needs of health care providers (HCPs) during challenging moments, the medical product assistant may provide reliable and up-to-date information in a variety of formats. For example, the medical product assistant may respond visually (e.g., text, images, gestures, etc.) and/or by voice. The medical product assistant may use voice activation and visual recognition to provide personalized access. The medical product assistant may provide information tailored to fit a user's specific requests (e.g., each healthcare professional). The medical product assistant may be used as a decision support tool that may provide suggestions (e.g., real-time suggestions) based on research, clinical evidence, and/or medical product information.

The medical product assistant may anticipate a customer's request and respond quickly and effectively. The medical product assistant may allow medical product manufacturers to meet the information requests of their customers more efficiently and effectively. For example, the medical product assistant may assist a user looking for product information, support, assistance, and/or product identification.

The medical product assistant may engage customers, HCPs, and/or patients. For example, the medical product assistant may provide a marketing approach focusing on delivering personalized and engaging customer experiences. This may involve identifying and prioritizing one or more customer groups, collecting data to understand their needs and interests better, and leveraging this information to deliver targeted marketing campaigns and other brand-building initiatives. The medical product assistant may also leverage technologies, such as visual and voice assistants, to provide a seamless experience for customers.

The medical product assistant may provide information for different audiences and/or contexts. The different audiences and/or contexts may include a user context, a user request context, a marketing context, and/or a business context.

The medical product assistant may address a user's target context. A healthcare provider may request the medical product assistant to provide information regarding clinical trials, such as phase one clinical trials. A healthcare provider may ask the medical product assistant to provide information regarding non-clinical trials, such as phase two non-clinical trials.

The medical product assistant may address a user request context. For example, the product assistant may handle customer requests in real-time. The medical product assistant may provide information for a customer, which may be a healthcare provider. The medical product assistant may provide healthcare stakeholders with a source of information that may increase their product awareness and improve their practice and performance. The medical product assistant may collect and/or provide clinical, economic, and usage information about medical products and/or medical programs.

The medical product assistant may address a marketing context. For example, the medical product assistant may engage healthcare providers through meaningful and interactive content. The content may provide information to healthcare providers that may increase engagement. The content may allow for the development of marketing leads and the adoption of medical products.

The medical product assistant may address a business contact. For example, the medical product assistant may enrich communications and interactions with current and new customers. The medical product assistant may assist in strengthening the brand of a medical product.

The medical product assistant may use and provide a voice-based interface. Given the rise in the popularity of voice-based interfaces, many users may prefer to use these technologies for completing work-related projects. A voice-based interface may provide increased convenience, accuracy, and efficiency. For example, using a voice assistant app, the medical product assistant, may allow a user to look up medical product information without manually entering data into a device, such as a phone or a computer. The medical product assistant may utilize machine learning capabilities and artificial intelligence, such as neural networks, to improve the accuracy of voice-enabled technologies, which may reduce the potential for transcription errors and misunderstandings. The medical product assistant may utilize natural language processing (NLP) algorithms to provide a voice interface that may be faster and more intuitive to use. The medical product assistant may utilize text-to-speech functionality (e.g., a speech synthesizer) to allow a device to read to a user. The medical product assistant may use speech recognition, allowing a device to listen and record the user's voice via a microphone so that it may be analyzed.

A medical product assistant may respond to user (e.g., patient or medical care provider) requests (e.g., using artificial intelligence). A medical product assistant may provide information to a user at an appropriate time (e.g., in real-time or on the detection of some condition). Such product information may be presented in one or more ways, including, for example, product recognition, package identification, and voice assistance. Product recognition may be provided in response to a photo (or, for example, a video feed) received by the medical product assistant. A medical product assistant may identify a product based on the image (or video feed). A medical product assistant may provide a presentation of a product that is recognized (e.g., a product recognition presentation). A product recognition presentation may include product information (e.g., model, date of manufacture, related products, product manual, tutorial information, etc.). The product recognition presentation may include commands available to the user, such as a command to order products (e.g., upgraded product versions, related accessories, refills, etc.), a command to provide a product simulator (e.g., via an applet), or other commands determined relevant (e.g., based on one or more of a user, a product, and context information).

A medical product assistant may support information acquisition (e.g., training materials) for a care provider. For example, a nurse may help a surgeon in performing a procedure. The procedure may be unfamiliar to the nurse. A medical product assistant may provide information associated with the procedure, such as identifying reloads (e.g., cartridges of staples) and associated products. A medical product assistant may enable product recognition, for example, by detecting and reading a QR code (e.g., found on a product box or on a product itself), recognizing packaging associated with a product, or in response to voice commands (e.g., product descriptions). In an example, a medical stapler may lock during a procedure. A medical product assistant may provide (e.g., in response to one or more of product recognition, voice commands, or commands provided in a product recognition presentation) instructions for performing a manual bailout of the locked stapler.

A medical product assistant may provide feedback for improving medical products. For example, a medical product assistant may be unable to provide the information requested by or relevant to a user. In such an example, the medical product assistant may provide feedback to the product manufacturer and/or supplier. The medical product assistant may include suggestions for improving the documentation or product design. The medical product assistant may use subsequent user activity to determine the appropriate information to provide in similar future cases.

By way of illustration, a medical product assistant may provide a manual for product operation and maintenance. The medical product assistant may determine that users (e.g., users identified with a particular care setting) regularly focus on a maintenance instruction in the manual, for example, an instruction describing product cleaning. The medical product assistant may provide feedback suggesting training resources (e.g., attention from a product salesperson and/or team), for example, in general, or to the particular care setting.

A medical product assistant may provide product information based on product packaging identification (e.g., a product box). Product information may be more recent or more detailed than the information available on the packaging. Product information may be interactive. For example, a medical product assistant may provide a user with access to an application (e.g., an applet) associated with the product identified.

A medical product assistant may use user information (e.g., identifying a user as a care provider, a product representative, or a product procurer) to determine relevant information relevant to the user. User information may be associated with information access permission. By way of illustration, a medical product assistant may determine whether to provide one user (e.g., a product representative) with product information that includes inventory status (e.g., whether a product is back-ordered), and may determine whether to provide another user (e.g., a surgeon) with product information that includes product operation instructions.

A medical product assistant may provide the information corresponding to products associated with an identified product (e.g., substitute products or competitor products). For example, a medical product assistant may identify an inferior product (e.g., a counterfeit or a competitor product that does not perform well) and provide product information indicating a superior product (e.g., a genuine article or a product that performs better than a competitor). For example, a medical product assistant may identify a known counterfeit product associated with a genuine product. For example, a medical product assistant may identify a product feature discrepancy (e.g., the presence or absence of a serial number, a product color of a product component) between an observed product and a known product and may identify the observed product as inferior (e.g., an older version of the known product or a counterfeit of the known product).

A medical product assistant may provide feedback based on the recognition of an inferior product. For example, a medical product assistant may associate recognition of an inferior product (e.g., a counterfeit) with location information (e.g., a GPS location provided by a device associated with the medical product assistant). For example, a medical product assistant may associate the recognition of one inferior product and the recognition of a second inferior product with correlation information. Such information may be provided, for example, to support further investigation of a potential counterfeit operation.

A medical product assistant may provide product information associated with the product, and/or packaging disposal. For example, product information may indicate that packaging is recyclable. For example, product information may indicate that one component of a device (e.g., a staple cartridge) may be regular waste, and another component of a device (e.g., a stapler head) may be medical waste. The disposal information may be provided as a live image, applying an indication of disposal instruction or category (e.g., a green indication, such as outlining for recyclable materials, and/or a red indication, such as highlighting for medical waste) to a scene representing identified products.

A medical product assistant may determine a confidence value associated with product identification. By way of illustration, two products may have visually similar body components (e.g., handles) and visually different control interfaces (e.g., knobs). A medical product assistant may identify a plurality of product candidates. The medical product assistant may provide information indicative of one or more candidate products. The medical product assistant may update a confidence value in response to receiving additional details regarding at least one of the two products.

A medical product assistant may base product identification on machine learning model(s). Such model(s) may be trained based on, for example, images captured in situ (e.g., by a conventional camera, a depth camera, and/or an infra-red camera, etc.), synthetic images based on computer models representing product geometry, computer models representing product geometry, or other data associated with product's physical properties.

A medical product assistant may present product information in an augmented-reality scene. For example, a scene including medical products may be augmented with information associated with the medical products, such as a plurality of labels, where a label may indicate a model of an identified product.

A medical product assistant may be used in a medical environment, such as an operating room, to help with various tasks related to using, training, and servicing the medical products being used. The information provided may include information regarding how to use the products, how to train others in their use, and/or how to troubleshoot any issues that may come up. The medical product assistant may be available during the pre-operative process, as well as during and after the surgery itself. This may help to ensure that everything runs smoothly, and that everyone involved knows what they are doing.

A medical product assistant may provide one or more ways to access information. For example, the medical product assistant may provide three ways to access information in a medical environment (e.g., an operating room): through voice commands, by pointing to the medical product itself, or by pointing to the box that the medical product came in. This may allow for quick and easy access to critical information during surgery.

A medical product assistant may provide one or more functionalities, such as package recognition, product recognition, and/or voice activation. The medical product assistant may make it easier and/or faster for users to get the information they need. The medical products assistant may be deployed using an app clip. An app clip may be a small, lightweight version of an app, such as the medical product assistant, which may be downloaded and used without installing the full app. App clips may be used to provide the medical product assistant (or a portion thereof) for users without requesting that a user download the entire medical product assistant.

The medical product assistant may provide a user with the information associated with the context in which the product may be used, found, purchased, and the like. The medical product assistant may provide a user with information based on the medical procedure performed or a stage of the medical procedure. The medical product assistant may be compatible with a smart device operating system, such as iOS, Android, and the like. The medical product assistant may use one or more APIs for object detection and/or image detection capabilities. The medical product assistant may use artificial intelligence that may be trained to detect a medical product using both real data and synthetic data.

The medical product assistant may be able to detect competitor devices, which may provide a level of insight into the competitive landscape. For example, the medical product assistant may perform an analysis of a competitor's device and may indicate opportunities for improvement. The medical product assistance may analyze a competitor's device to provide data for marketing and sales efforts.

The medical product assistant may determine a competitor's product and may show a medical product that competes with the competitor's product to the user. The medical product assistant may indicate why the medical product may be better than a competitor product. The medical product assistant may reveal one or more advantages the medical product may have over competitor products.

Machine learning algorithms may be trained by feeding them data (e.g., a lot of data), which may be in the form of 3D models, polygons, images, and/or photorealistic images. The more data may be provided, the smarter the machine-learning algorithm may become.

Synthetic data may be used to train machine learning algorithms. For example, the medical product assistant may use and/or may create synthetic data to train MLS models. This may be done, for example, to provide data that may be more accurate, quicker to produce, and more cost-effective than other methods of generating data, such as hand annotating imagery.

The accuracy of photorealistic synthetic data may be varied and/or may be selected to improve machine learning algorithms. The accuracy of photorealistic synthetic data may depend on the specific application.

Training data may include creating data that may be obscured or may not be easily visible. This may be done by, for example, presenting half of an object instead of the whole object (e.g., half an apple instead of the whole fruit). This may allow the machine learning algorithm to focus on how to identify the object and how to ignore background noise. Synthetic data may be created using deep vision data.

Synthetic data may be helpful with the annotation of 3D models. Synthetic data may allow for the ability to create data in an automated fashion and randomize it to create different camera angles, shots, colors, and the like.

There are multiple ways to generate training data. Training data may be generated using neural radiance fields. This may involve feeding the algorithm a sparse set of images and training it to create a reverse-rendered dataset. Training data may be generated using a generative adversarial network (GAN) or 3D GaNS to create 3D shapes. For example, to train an algorithm to recognize the shape of a skeletonized colon, a number of different 3D shapes of a skeletonized colon may be generated. Training data may be generated using domain randomization. For example, domain randomization may be used to train a machine-learning algorithm on the shape of an object package and the branding on the package. This may be done, for example, to reduce concern regarding background noise or other objects in the scene.

Using synthetic data may help train artificial intelligence models quickly without having to worry about data being perfect and/or correct. With other data, such as real data, it may take more time to annotate pictures and make sure everything is perfect or correct. By using synthetic data, it may be possible to iterate quickly and make changes without spending as much time upfront.

Images and/or CAD models may be modified for artificial intelligence training purposes. For example, the images and/or CAD models may be modified to simulate different lighting conditions such that artificial intelligence models may learn from the different lighting conditions and create better results.

The medical product assistant may be able to use one or more AI/ML models to determine if a product may be a counterfeit or if the product may have an anomaly. The medical product assistant may determine that the logo detected on a product may be of the wrong color. The medical product assistant may determine that there may be a mark on the product that may be in an incorrect location or may have an incorrect color, size, or shape.

The medical product assistant may determine that a color, size, or shape of a product may be incorrect. The medical product assistant may determine that a portion of the medical product assistant may be incorrect (e.g., such as the shape of the product handle). The medical product assistant may determine that a product package may be incorrect. The medical product assistant may determine that the product may have an anomaly. The medical practice system may determine that the product may be a counterfeit.

An anomaly detection model and/or a counterfeit detection model may be trained with images of a product that may be designed to determine how different an object (e.g., along a scale) may be when compared to the medical product. An anomaly detection model and/or a counterfeit detection model may use color, infrared, or ultraviolet (UV) to help determine if an object may be counterfeit. For example, the color may be a color that may be detected by an infrared sensor but may not be detected by the human eye.

The medical product assistant may use other methods for detection in conjunction with or as a standalone method for detecting counterfeit devices. These may include watermarks, special inks, micro-printing, raised printing, holograms, and/or barcodes. Combining these methods may increase the chances of correctly detecting a counterfeit device.

A medical product detection model, an anomaly detection model, and/or a counterfeit detection model may use signal data, such as Bluetooth data, to recognize a medical product. For example, Bluetooth data collected from a device may be used to train a neural network. Bluetooth data may include data from the motor of the device. Artificial intelligence models may compare the collected data against known data for an identified medical product to determine if the object producing the collected data, is the medical product. If the object may be determined to be a medical product, then medical product data (e.g., product information) may be provided to a user.

The medical product assistant may be able to communicate with a medical product, such as a medical device. The medical product assistant may be able to determine the information that may be useful for preventing a risk during surgery. For example, the medical product assistant may communicate with a surgical stapler to provide a surgeon with information about tissue plowing or other potential surgery risks. The medical product assistant may provide the surgeon, nurse, or scrub tech feedback about what was done well and what may be improved. This may help ensure that surgeons have up-to-date information to make better patient decisions.

The medical product assistant may allow the user to use voice commands. The medical product assistant may allow one or more medical products to become voice activated. For example, the medical product assistant may enable the user to control a medical product using voice commands.

Voice recognition may be integrated into the medical product assistant so that the medical product assistant may respond to a user using voice commands. A user may engage in a conversation with the medical product assistant, and the medical product assistant may introduce products, marketing material, and/or services to the user.

The medical product assistant may provide a link to our website, where a user may enter product codes and create a PDF with barcodes they would like to print out. This may allow the user to go to a central supply and pull the products that they may need.

The medical assistant may keep track of inventory levels. If a product has been used in a hospital, the medical product may track the number of available products so that a user may not have to count how many are left on the shelf. This may prevent a user from running out of a medical product used for a medical procedure.

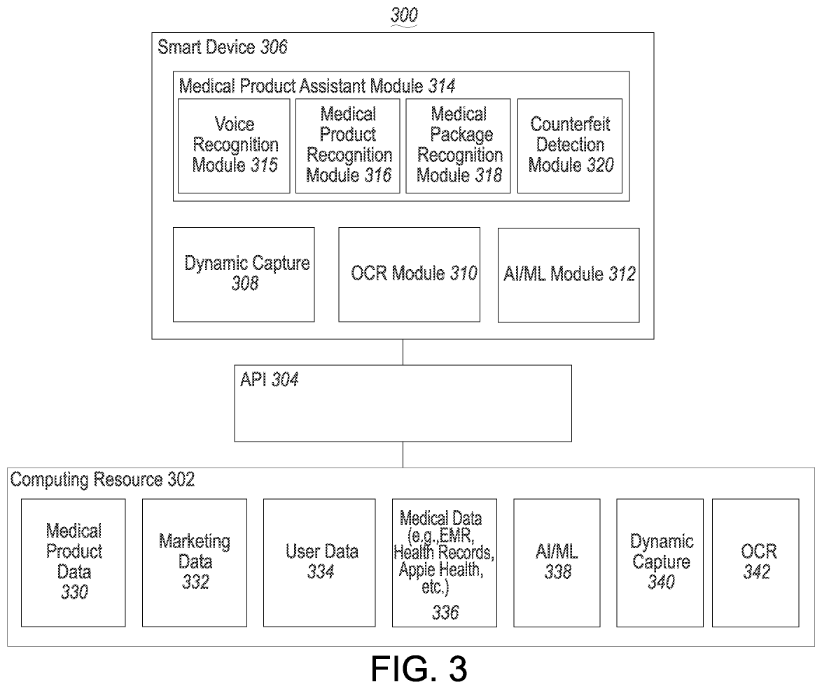

FIG. 3 depicts a system block diagram that may include one or more modules (e.g., software modules) for providing a medical product assistant to recognize, identify, and support a medical product. As shown in FIG. 3, system 300 may include computing resource 302, APIs 304, and smart device 306. Smart device 306 may be a smartphone, a tablet (e.g., an iPad), a smartwatch, a wearable device, a cellular phone, a computer, a server, and the like. For example, smart device 306 may be a smart device that may include components 120, shown in FIG. 1. Smart device 306 may be smartwatch 206, smartphone 204, or computing resource 212 shown in FIGS. 2A-B. Computing resource 302 may be computing resource 212 shown in FIGS. 2A-2B.

Referring to FIG. 3, smart device 306 may comprise one or more software modules, such as dynamic capture module 308, optical character recognition (OCR) 310, artificial intelligence/machine learning (AI/ML) 312, and/or medical product assistant module 314.

Medical product assistant module 314 may provide information tailored to fit a user's specific requests (e.g., each healthcare professional). Medical product assistant module 314 may be used as a decision support tool that may provide suggestions (e.g., real-time suggestions) that may be based on research, clinical evidence, and/or medical product information. Medical product assistant module 314 may include voice recognition module 315, medical product recognition module 316, medical package recognition module 318, and/or counterfeit detection module 320.

Although not shown in FIG. 3, medical product assistant module 314 may be hosted by computing resource 302. For example, medical product assistant module 314 may be offloaded to computing resource 302, and smart device 306 may utilize the functionality provided by medical product assistant module 314. Similarly, one or more modules of medical product assistant module 314 may be hosted by computing resource 302. For example, voice recognition module 315, medical product recognition module 316, medical package recognition module 318, and/or counterfeit detection module 320 may be offloaded to computing resource 302.

Voice recognition module 315 may allow medical product assistant module 314 to receive and/or respond to a user's voice. For example, medical product assistant module 314 may receive a request from a user. The request may include a sound recording with a verbal command and/or verbal inquiry from a user. Medical product assistant module 314 may provide sound recording to voice recognition module 315. Voice recognition module 315 may analyze the sound recording to retrieve the user's verbal command and/or verbal inquiry. Voice recognition module 315 may provide verbal commands and/or verbal inquiries to medical product assistant module 314. Medical product assistant module 314 may use verbal inquiry and/or commands to determine that the user may be requesting information regarding a medical product. Medical product assistant module 314 may provide medical product information to the user.

Voice recognition module 315 may allow a user to look up medical product information without manually entering data (e.g., entering data by touching the device) into a device (e.g., a smart device), such as a smartphone or a computer. Voice recognition module 315 may utilize machine learning capabilities and artificial intelligence (e.g., provided by AI/ML module 312), such as neural networks, to improve the accuracy of voice-enabled technologies, which may reduce the potential for transcription errors and/or misunderstandings. Voice recognition module 315 may utilize natural language processing (NLP) algorithms to provide a voice interface that may be faster and/or more intuitive. Voice recognition module 315 may utilize text-to-speech functionality (e.g., a speech synthesizer) to allow a device to read to a user. The medical product assistant may use speech recognition, allowing a device to listen and/or record the user's voice via microphone so that it may be analyzed. Voice recognition module 315 may provide Automatic Speech Recognition (ASR), which may allow users to use their voices to speak with a computer interface that may resemble a human conversation.

Medical product recognition module 316 may recognize and/or identify a medical product. For example, medical product recognition module 316 may determine a medical product from a photo and/or a video. The medical product may be a medical instrument, a medical device, medical equipment, a medication, and the like.

Medical product recognition module 316 may provide an identifier for the medical product. The identifier may include, for example, a text description of the medical product, a medical product manufacturer, a model number of the medical product, and the like.

Medical product recognition module 316 may identify a medical product using a photo of the medical product. For example, medical product recognition module 316 may retrieve an image, identify a portion of an object within the image, and determine that the portion of the object is associated with the medical product. For example, within an image, the object may be partially obstructed, such that a first portion of the object is hidden, and a second portion of the object is exposed. Medical product recognition module 316 may recognize that the second portion of the object is a portion of a medical product, such as a shaft of a surgical stapler, and may recognize the object as a surgical stapler. The determination that the portion of the object is associated with the medical product may be based on a confidence level or score. The medical product recognition module 316 may determine that the confidence level or score is above a threshold.

Medical product recognition module 316 may retrieve a video of the medical product. For example, medical product recognition module 316 may receive video data (e.g., from a user device) and/or access video data (e.g., from a video source). Medical product recognition module 316 may process the video data to identify one or more frames that may include the medical product. In examples, medical product recognition module 316 may identify the medical product in real-time as the video is collected and/or played.

If medical product recognition module 316 identifies the medical product in the image and/or video, medical product recognition module 316 may provide an identifier for the medical product. The identifier provided by medical product recognition module 316 may include a text description of the medical product. The text description of the medical product may be retrieved from a database (e.g., a knowledge database) based on the determined identity of the medical product.

The medical product recognition module 316 may use synthetic data to recognize a medical product or a portion of a medical product. Synthetic data may be algorithmically generated data that imitates real data and may substitute for datasets used for modeling and training in artificial intelligence. Medical product recognition module 316 may generate one or more synthetic images of the medical product from image data, video data, and/or computer-automated drawings (CAD models). As described herein, synthetic data (e.g., images) may be used to train a machine learning model to recognize the medical product in the image and/or video data.

Medical product recognition module 316 may output data associated with the recognized medical product. Medical product recognition module 316 may provide an identifier for the recognized medical product (e.g., may send the identifier to medical product assistant module 314). The identifier for the recognized medical product may include, for example, a text description of the medical product, a medical product manufacturer, a model number of the medical product, and the like.

The identifier provided by medical product recognition module 316 may include medical product information, such as manufacturer information, for the medical product. As described herein, the medical product information may be retrieved from a database based on the determined identity of the medical product.

Medical product recognition module 316 may identify a portion of a medical product within an image and may use that portion to determine the identity of the medical product. Medical product recognition module 316 may identify a label on the medical product. The label may include text and/or images that describe the medical product. The label may be a barcode, QR code, and the like. Medical product recognition module 316 may use optical character recognition (OCR) to process the text on the label and determine information about the medical product from the text.

Medical product recognition module 316 may identify an image on the medical product. The image may be a logo of the manufacturer of the medical product, an image of the medical product, and the like. Medical product recognition module 316 may compare the identified image with images in one or more databases as described herein.

The shape of the medical product may be used to identify the medical product. For example, a medical product may be recognized based on one or more edges in an image of the medical product. One or more edges in the image of the medical product may be identified using a machine-learning model as described herein.

The color of the medical product may be used to identify the medical product. The color of the medical product may be determined from an image of the medical product. The color of the medical product may be compared with colors associated with known medical products to identify the medical product.

Medical product recognition module 316 may identify a medical product based on color. For example, a medical product may be provided in a standardized color (e.g., white for a handle of a surgical stapler, green for a cartridge of surgical staples). The identification of standardized color may be used to identify a medical product. The color may be a color that may be detected by an infrared sensor but may not be seen by the human eye.

The size of the medical product may be used to identify the medical product. The size of the medical product may be determined using one or more images of the medical product. The size of the medical product may be compared with sizes associated with known medical products to identify the medical product.

A combination of information about the medical product (e.g., shape, color, size, label, and/or image) may be used to determine the identity of the medical product. For example, a machine learning model may be trained to recognize medical products based on a combination of the medical product's shape, color, size, label, and/or image.

A label, and/or an identifier, for a medical product, may be used to identify a medical product. For example, a medical product may include text, and/or images, which identify the medical product. In examples, medical product recognition module 316 may use OCR, (e.g., via OCR 310 and/or OCR 342) to process text on the medical product and determine information about the medical product from the processed text.

Medical product recognition module 316 may provide data associated with the recognized medical product to medical product assistant module 314. Medical product assistant module 314 may use the data to provide a user with information about the recognized medical product. Medical product assistant module 314 may provide information about how to use the recognized medical product, possible side effects of using the recognized medical product, warnings associated with using the recognized medical product, and/or drug interactions associated with using the recognized medical product.

Medical product recognition module 316 may use one or more machine learning models trained to recognize medical products. The machine learning models may be trained using images and/or videos of known medical product packaging. The machine learning models may be convolutional neural networks (CNNs).

Medical package recognition module 318 may recognize and/or identify a medical product package. For example, medical package recognition module 318 may determine a medical product package from a photo and/or a video. The medical product package may be a package and/or wrapping associated with a medical instrument, a medical device, medical equipment, medication, and the like.

Medical package recognition module 318 may be used to provide an identifier for the medical product package. The identifier may include, for example, a text description of the medical product package, a manufacturer of the medical product package, a model number of the medical product package, and the like.

The medical package recognition module 318 may recognize a medical product package from an image and/or a video. Medical package recognition module 318 may identify a box, bottle, blister pack, and the like that includes one or more doses of a medication product. In examples, medical package recognition module 318 may be combined with the medical product recognition module 316 to determine an identifier for the medical product.

Medical package recognition module 318 may identify a medical product using a photo of the medical product package. For example, medical package recognition module 318 may retrieve an image, identify a portion of an object within the image, and determine that the portion of the object is associated with the medical product package. For example, within an embodiment, the object may be partially obstructed, such that a first portion of the object is hidden, and a second portion of the object is exposed. Medical package recognition module 318 may recognize that the second portion of the object is a portion of a medical product package, such as a box for a surgical stapler and may recognize the object as the box of the surgical stapler. The determination that the portion of the object is associated with the medical product package may be based on a confidence level or score. Medical package recognition module 318 may determine that the confidence level or score may be above a threshold.

Medical package recognition module 318 may retrieve a video of the medical product package. For example, medical package recognition module 318 may receive video data (e.g., from a user device) and/or access video data (e.g., from a video source). Medical package recognition module 318 may process the video data to identify one or more frames that may include the medical product package. In examples, medical package recognition module 318 may identify the medical product package in real-time as the video is collected and/or played.

If medical package recognition module 318 identifies the medical product package in the image and/or video, medical package recognition module 318 may provide an identifier for the medical product. The identifier provided by medical package recognition module 318 may include a text description of the medical product package. The text description of the medical product package may be retrieved from a database (e.g., a knowledge database) based on the determined identity of the medical product.

Medical package recognition module 318 may use synthetic data to recognize a medical product package or a portion of a medical product package. Synthetic data may be algorithmically generated data that imitates real data and may substitute for datasets used for modeling and training in artificial intelligence. Medical package recognition module 318 may generate one or more synthetic images of the medical product package from image data, video data, and/or computer-automated drawings (CAD models). As described herein, the synthetic data (e.g., images) may be used to train a machine learning model to recognize the medical product package in the image and/or video data.

Medical package recognition module 318 may output data associated with the recognized medical product package. Medical package recognition module 318 may provide an identifier for the recognized medical product package and/or medical product (e.g., provide the identifier to medical product assistant module 314). The identifier for the recognized medical product may include, for example, a text description of the medical product, a medical product manufacturer, a model number, and the like.

The identifier provided by medical package recognition module 318 may include medical product information, such as manufacturer information, for the medical package and/or medical product. As described herein, the medical product information may be retrieved from a database based on the determined identity of the medical product.

Medical package recognition module 318 may identify a portion of a medical product package within an image and may use that portion to determine the identity of the medical product package and/or medication product. Medical package recognition module 318 may identify a label on the medical product package. The label may include text and/or images that describe the medical product package. The label may be a barcode, QR code, and the like. Medical package recognition module 318 may use optical character recognition (OCR) to process the text on the label and determine information about the medical product package from the text.

Medical package recognition module 318 may identify an image on the medical product package. The image may be a logo of the manufacturer of the medical product package, an image of the medical product package, and the like. Medical package recognition module 318 may compare the identified image with images in one or more databases described herein.

The shape of the medical product package may be used to identify the medical product package and/or the medical product. For example, a medical product may be recognized based on one or more edges of an object in an image. One or more edges in the image may be identified using a machine-learning model described herein.

The color of the medical product package may be used to identify the medical product package and/or the medical product. The color of the medical product package may be identified from an image of the medical product package. The color of the medical product package may be compared with colors associated with known medical product packages to identify the medical product package and/or the medical product.

The medical package recognition module 318 may identify a medical product package and/or a medical product based on a color associated with a medical product package. For example, a medical product package may be provided in a standardized color (e.g., white for a box of the package, red for the text of the package, etc.). The identification of the medical product package with the standardized color may be used to identify the medical product. The color may be a color that may be detected by an infrared sensor but may not be seen by the human eye.

The size of the medical product package may be used to identify the medical product package and/or the medical product. The size of the medical product package may be identified using one or more images of the medical product package. The size of the medical product package may be compared with sizes associated with known medical product packages to identify the medical product package and/or the medical product.

A combination of information about the medical product package (e.g., shape, color, size, label, and/or image) may be used to determine the identity of the medical product package and/or medical product. For example, a machine learning model may be trained to recognize medical product packages (e.g., and their contents, such as medical products) based on a combination of the shape, color, size, label, and/or image of the medical product package.

A label and/or an identifier for a medical product package may be used to identify a medical product package and/or a medical product. For example, a medical product package may include text and/or images that identify the medical product. In examples, medical package recognition module 318 may use OCR (e.g., via OCR 310 and/or OCR 342) to process text on the medical product and determine information about the medical product from the processed text.

Medical package recognition module 318 may provide data associated with the recognized medical product package and/or medical product to medical product assistant module 314. Medical product assistant module 314 may use the data to provide a user with information about the recognized medical product. Medical product assistant module 314 may provide information about how to use the recognized medical product, possible side effects of using the recognized medical product, warnings associated with using the recognized medical product, and/or drug interactions associated with using the recognized medical product.

Medical package recognition module 318 may use one or more machine learning models trained to recognize medical product packaging. The machine learning models may be trained using images and/or videos of known medical product packaging. The machine learning models may be convolutional neural networks (CNNs).

Counterfeit detection module 320 may be configured to detect counterfeit medical products. The counterfeit detection module 320 may receive data associated with a medical product package from medical package recognition module 318 and/or data related to a medical product from medical product recognition module 316. Counterfeit detection module 320 may use the data to determine whether the medical product is counterfeit.

The counterfeit detection module 320 may use one or more machine learning models to determine whether the medical product is counterfeit. Machine learning models may be trained using images and/or videos of known counterfeit medical products and/or known genuine medical products. The machine learning models may be convolutional neural networks (CNNs).

Counterfeit detection module 320 may use synthetic data to detect counterfeit medical products. In examples, features learned or extracted from synthetic data may be transferred to real data to improve the performance of a machine learning model on data (e.g., real data). For example, a machine learning model may be trained using synthetic images of medical devices that include known good and bad examples of the medical device (e.g., genuine vs. counterfeit). The machine learning model may then be applied to images (e.g., real images) to predict whether the medical devices in the images are genuine or counterfeit.

Counterfeit detection module 320 may provide data on whether the medical product is counterfeit to the medical product assistant module 314. Medical product assistant module 314 may use the data to provide information about whether the medical product is a counterfeit product to a user.

Dynamic capture module 308 may capture an image, a video, and the like. Dynamic capture module 308 may provide image and/or video processing. For example, smart device 306 may use dynamic capture module 308 to capture an image of a medical product and/or a medical product package, which may be used to determine the identity of the medical package, the medical product, and/or other information about the medical product.

Dynamic capture module 308 may allow a user to point a camera of smart device 306 at a medical product, a medical product package, a medical product label, and the like to capture information regarding the medical product without having the user record an image or video.

Dynamic capture module 340 may provide the functionality provided by dynamic capture module 308. Dynamic capture module 340 may be located on computing resource 302. Dynamic capture module 340 may allow smart device 306 to offload the capabilities of dynamic capture module 308 to computing resource 302.

OCR module 310 may determine text and/or other information from an image or video. OCR module 310 may determine the text on an image of a medical product and/or a medical product package. OCR module 310 may be used to determine the text on an image of a medical product label, a portion of a medical product label, a medical product, a portion of a medical product, a medical product package, a portion of a medical product package, a combination thereof, and the like. The text may be used to determine information regarding the medical product. The text may include identifying information for the medical product, such as a medical product identifier, a brand name, a model name, a name for the medical product, medical product information that may be included on the medical product package, and the like.

OCR module 342 may provide the functionality provided by OCR 310. OCR 342 may be located on computing resource 302. OCR module 342 may allow smart device 306 to offload the capabilities of OCR module 310, to computing resource 302.

AI/ML module 312 may provide artificial intelligence (AI) and/or machine learning (ML) models, services, and the like. AI/ML module 312 may be integrated with or otherwise communicate with one or more of the other modules described herein, such as voice recognition module 315, medical product recognition module 316, medical package recognition module 318, counterfeit detection module 320, and/or medical product assistant module 314.

AI/ML module 312 may provide a machine learning model used by medical product recognition module 316 to identify a medical product from image and/or video data. AI/ML module 312 may provide a machine learning model used by counterfeit detection module 320 to determine whether a medical product is counterfeit. AI/ML module 312 may provide a machine learning model used by medical product assistant module 314 to provide information about one or more medical products.

AI/ML module 312 may include machine learning, a branch of artificial intelligence that seeks to build computer systems that may learn from data without human intervention. These techniques may rely on creating analytical models that may be trained to recognize patterns within a dataset. The dataset may include data and/or synthetic data. The data may include images of a medical product, video of a medical product, data describing a medical product, images of a medical product package, video of a medical product package, data describing a medical product package, images of a medical product label, video of a medical product label, data describing a medical product label, and the like. As described herein, the synthetic data may be generated based on the data. For example, synthetic data may include images of a medical product manipulated to include different lighting conditions, backgrounds, angles, and the like.

The synthetic data may include images of a medical product where the background may be a medical setting, such as an operating room, a doctor's office, a hospital, and the like. The synthetic data may also include images of a medical product where the medical product has been placed in different positions, such as on a table, on a shelf, held by a person, and the like.

AL/ML module 312 may include one or more models. These models may be deployed to apply these patterns to data, such as medication labels, to improve the identification of medication. AI/ML module 312 may include supervised machine learning, unsupervised machine learning, reinforcement learning, and/or cognitive computing (CC). For example, AI/ML module 312 may use cognitive computing (CC) to utilize one or more self-teaching algorithms that may use data mining, visual recognition, voice recognition, and/or natural language processing to identify a medical product and/or packaging associated with the medical product.

AI/ML 338 may provide the functionality of AI/ML module 312. AI/ML 338 may be located on computing resource 302. AI/ML 338 may allow smart device 306 to offload the capabilities of AI/ML module 312 to computing resource 302.

API 304 may allow smart device 306 to send and/or receive data from computing resource 302. For example, API 304 may include an application programming interface (API) that may allow smart device 306 to send data to and/or receive data from computing resource 302. API 304 may include a web API, such as a representational state transfer (REST) API, which may use hypertext transfer protocol (HTTP) requests and responses to allow smart device 306 to send data to and/or receive data from computing resource 302. API 304 may use other protocols such as simple object access protocol (SOAP), advanced message queuing protocol (AMQP), and the like.

API 304 may include a secure sockets layer (SSL) that may encrypt data sent between smart device 306 and computing resource 302. API 304 may include a transport layer security (TLS) that may encrypt data sent between smart device 306 and computing resource 302. The encryption provided by SSL and/or TLS may provide for data confidentiality and/or data integrity.

Computing resource 302 may include one or more resources such as medical product data 330, marketing data 332, user data 334, medical data 336, AI/ML 338, dynamic capture module 340, OCR 342, a combination thereof, and the like.

Medical product data 330 may be data stored in or may be a database. Medical product data 330 may include data associated with one or more medical products. For example, medical product data 330 may include a database of medication names, active ingredients, dosages, indications, contraindications, warnings, and the like.

Medical product data 330 may be stored on a remote computing resource that may be accessed by computing resource 302 via a network, such as the Internet. Medical product data 330 may be stored on a local computing resource. Medical data 332 may include images and/or videos of medical products, text describing medical products, and the like. Medical product data 330 may include images and/or videos of medical product packaging, text describing medical product packaging, and the like.

Medical product data 330 may train AI/ML models for counterfeit detection. For example, AI/ML models may be trained to identify features indicative of a genuine medical product instead of a counterfeit medical product.

Medical product data 330 includes synthetic data for one or more medical products and/or one or more medical product packages. Synthetic data may be generated by a computer program and may be used to train AI/ML models. Synthetic data may be generated by a computer program configured to generate data similar to real data. Synthetic data may be generated for a variety of reasons, such as to supplement accurate data when there is not enough real data available, to protect the privacy of individuals whose data is being used to train AI/ML models, and the like.

Marketing data 332 may be data or may be a database. Marketing data 332 may include data associated with marketing campaigns. For example, marketing data 332 may include databases of customer names, customer contact information, customer demographics, campaign materials, and the like.

User data 334 may be data or may be a database. User data 334 may include data associated with one or more users of smart device 306. For example, user data 334 may include databases of usernames, user contact information, user demographics, and the like. User data 334 may include data that may have been generated by a smart device, such as smart device 306. For example, smart device 306 may generate data regarding the usage of smart device 306, such as the frequency with which certain features are used, the duration of use of certain features, and the like.

Medical data 336 may be data or may be a database. Medical data 336 includes data from an EMR, a health record, an Apple Health record, and the like. Medical data 336 may include patient data, provider data, health insurance data, clinical trial data, and the like. Patient data may include demographic information (e.g., age, gender, race), contact information (e.g., address, email, phone number), medical history, family medical history, and the like. Provider data may include provider demographic information (e.g., age, gender, race), contact information (e.g., address, email, phone number), specialty, board certifications, and the like. Health insurance data may include insurance carrier, policy number, group number, and the like. Clinical trial data may include the name of the clinical trial, the sponsor of the clinical trial, eligibility criteria for participation in the clinical trial, and the like.

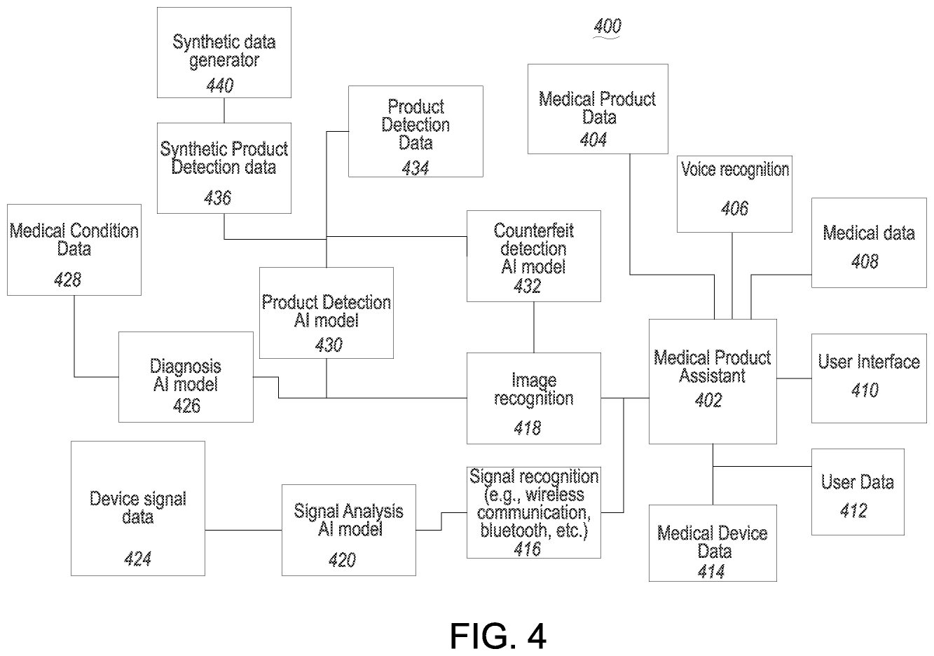

FIG. 4 depicts a block diagram of a system for collecting data and/or training artificial intelligence (AI) to be used by a medical product assistant.

System 400 may provide, train, host, deploy, and/or generate medical product assistant 402. System 400 may include one or more computing resources and/or smart devices, such as server(s), client(s), database(s), application(s), and/or the like. Computing resource(s) may be communicatively coupled via a network, which may be any network, such as the Internet, an intranet, a local area network (LAN), a wide area network (WAN), a wireless network, and/or the like. The computing resources may include hardware and/or software for executing one or more instructions. The computing resources may be configured to execute one or more computer programs, which may be used to provide, train, host, deploy, and/or generate medical product assistant 402. For example, a computing resource may be used to provide medical product data 404, voice recognition 406, medical data 408, user interface 410, user data 412, medical device data 414, signal recognition 416, and/or image recognition 418.

Medical product assistant 402 may be a software application configured to provide information regarding medical products, such as medical devices. Medical product assistant 402 may include and/or may use an AI/ML model that may have been trained using data from one or more data sources, such as medical product data 404, voice recognition 406, medical data 408, user interface 410, user data 412, medical device data 414, signal recognition 416, and/or image recognition 418.

Medical product assistant 402 may be configured to provide information in response to one or more questions posed by a user of medical product assistant 402. For example, a user may ask medical product assistant 402 to provide information regarding a particular type of medical device. In response, medical product assistant 402 may provide information about the medical device, such as a description of how to use the medical device, warnings and/or precautions associated with the medical device, and the like. In examples, medical product assistant 402 may be configured to provide information about a plurality of medical devices. For example, if a user asks medical product assistant 402 for information about available types of surgical staplers, medical product assistant 402 may provide information about one or more types of surgical staplers.

Medical product assistant 402 may be used by one or more healthcare providers to obtain information about medical products. Examples of some of the questions medical product assistant 402 may address may be as follows:
What is the procedure for disposing of this device after a medical procedure?
How should this device be cleaned after a medical procedure?
Can you provide instructions for assembling this device?
What are the recycling instructions for this device?
What are the disposal instructions for this device?

Medical product assistant 402 may be asked to provide information about a user's location (e.g., current location). The medical product assistant 402 may use the GPS on a user's mobile device to determine the user's current location. Medical product assistant 402 may use the user's current location to provide information about medical products available in the user's current location.

Medical product assistant 402 may be asked to provide information about a medical product that is available in a particular location. The medical product assistant 402 may use the GPS on a user's mobile device to determine the user's current location. Medical product assistant 402 may use the user's current location to provide information about medical products available in the user's current location.

Medical product assistant 402 may be asked to provide information about a medical product available online. Medical product assistant 402 may link to a website where the medical product is available for purchase.

Medical product assistant 402 may provide a list of medical products. The list of medical products may include, for example, one or more of the medical devices (e.g., all the medical devices) that a particular manufacturer makes, one or more of the types of medical devices that may be used to treat a particular condition, one or more of the types of medical devices that may be suitable for a particular age group, one or more of the types of medical devices that may be suitable for a particular gender, and the like. If a list of medical products may be provided, the medical product assistant may receive the selection of a medical product from the list of medical products.

Medical product assistant 402 may provide instructions for using a medical product. The instructions may include, for example, how to use the medical device, how to properly care for the medical device, how often to use the medical device, when to replace the medical device, and the like.

Medical product assistant 402 may be used by a surgeon to obtain information about a particular type of medical device during surgery. A healthcare provider may use medical product assistant 402 to request instructions on cleaning a surgical stapler (e.g., a medical product) after a surgical procedure. The medical product assistant may be asked to provide post-operative instructions for a medical device.

A healthcare provider may use medical product assistant 402 to request instructions on how to dispose of a medical product after a medical procedure. Medical product assistant 402 may provide information regarding the medical product's disposal, recycling, cleaning, and/or disassembly.

Medical product assistant 402 may be used by a healthcare provider to determine if a particular medical device may be compatible with a particular type of medication. A healthcare provider may use medical product assistant 402 to request instructions on dispensing medication (e.g., a medical product).

Medical product assistant 402 may be asked to provide information about a medical product recall. The medical product assistant 402 may provide this information in real-time as product recalls are announced.

Medical product assistant 402 may be used by one or more patients to obtain information about medical products. For example, medical product assistant 402 may be used by a patient to get information about a type of medical device that the patient may be considering for a medical procedure. A patient may use medical product assistant 402 to request instructions on how to dispose of a medical product after a medical procedure.

A patient may use medical product assistant 402 to request instructions for follow-up care and/or a diagnosis. For example, a patient may ask if a surgical site is healing correctly and provide a photo of the site to medical product assistant 402. Medical product assistant 402 may analyze the image to determine if the patient may be healing correctly and may provide follow-up instructions regarding the surgical site.

Medical product assistant 402 may be used by one or more caretakers to obtain information about medical products. For example, medical product assistant 402 may be used by a caretaker to get information about a type of medical device that the caretaker's patient may be using. A caretaker may use medical product assistant 402 to request instructions on how to dispose of a medical product after a medical procedure.

Medical product assistant 402 may be used by one or more insurance providers to obtain information about medical products. For example, medical product assistant 402 may be used by an insurance provider to determine if an insurance policy covers a particular type of medical device. A patient and/or a healthcare provider may use medical product assistant 402 to request instructions on filing a claim with an insurance provider for a medical device.

Medical product assistant 402 may be used by one or more research organizations to obtain information about medical products. For example, a medical product assistant 402 may be used by a research organization to determine if a particular type of medical device may be suitable for a particular study.

Medical product assistant 402 may provide a medical product recommendation. Medical product assistant 402 may be asked to provide information about the types of users requesting information about a medical product.

Medical product assistant 402 may be asked to identify a medical product. This may be done with a picture, a video, a description, a voice command, a combination thereof, and the like. Medical product assistant 402 may identify a medical product using an image that may include the medical product, a portion of the medical product, a package associated with the medical product, a portion of the package associated with the medical product, a combination thereof, and the like. Medical product assistant 402 may identify the medical product from a database of images of medical products. In examples, medical product assistant 402 may use artificial intelligence (“AI”) to identify the medical product from the image.

Medical product assistant 402 may be asked to determine if a medical product is counterfeit. In examples, medical product assistant 402 may automatically make this determination and warn a user that a medical product is counterfeit. In examples, medical product assistant 402 may provide information to help a user determine if a medical product is counterfeit. For example, the medical product assistant may provide information about where and when a medical product was manufactured.

Medical product assistant 402 may provide a notification in response to determining that a medical product is counterfeit. For example, after analyzing an image of a surgical stapler with image recognition 418 and determining that the surgical staple is counterfeit, medical product assisted 402 may provide a notification. The notification may indicate that the surgical stapler is counterfeit and should not be used. The notification may be provided to a user of the medical product.

Medical product data 404 may be accessed by medical product assistant 402 to provide information about medical products. Medical product data 404 may include data regarding medical products, such as medical devices. The medical product data 404 may include, for example, a catalog of medical devices, device specifications, instructions for use (IFU), and the like. Medical product data 404 may be collected from one or more data sources, such as manufacturers, distributors, retailers, and the like. Medical product data 404 may include product information (e.g., name, manufacturer, model number), safety information (e.g., warnings, precautions), indications for use, contraindications, instructions for use, and/or troubleshooting information, warranty information, and the like.

Voice recognition 406 may be accessed by medical product assistant 402 to convert speech into text. Voice recognition 406 may include, for example, automatic speech recognition (ASR) and the like. In examples, voice recognition 406 may convert speech into text and provide information about a particular medical device.

Voice recognition 406 may be used to receive input from a user. For example, voice recognition 406 may be used to receive a question from a user and/or to receive input regarding a particular medical product. Voice recognition 406 may be configured to convert the received input into text. For example, voice recognition 406 may use natural language processing to process the received input and may determine the meaning of the received input. For example, voice recognition 406 may be used to identify one or more keywords in the received input and/or determine the context of the received input. As an example, natural language processing may be used to identify key terms in the text, such as medical product names, and/or to convert the identified key terms into a format that may be searched.

Medical data 408 may be accessed by medical product assistant 402. Medical data 408 may include data from an EMR, a health record, an Apple Health record, and the like. Medical data 408 may include patient data, provider data, health insurance data, clinical trial data, and the like. Patient data may include demographic information (e.g., age, gender, race), contact information (e.g., address, email, phone number), medical history, family medical history, and the like. Provider data may include provider demographic information (e.g., age, gender, race), contact information (e.g., address, email, phone number), specialty, board certifications, and the like. Health insurance data may include insurance carrier, policy number, group number, and the like. Clinical trial data may include the name of the clinical trial, the sponsor of the clinical trial, eligibility criteria for participation in the clinical trial, and the like.

Medical data 408 may include images and/or videos of medical procedures, text describing medical procedures, and or the like.

User interface 410 may be accessed by medical product assistant 402. User interface 410 may include one or more user interfaces configured to receive input from a user and to provide output to the user. User interface 410 may be an interface that may be used with a smart device, a smartwatch, a smartphone, a personal computer, a laptop, a server, a combination thereof, and the like.

User data 412 may include data associated with one or more users of smart device 306. For example, user data 412 may include databases of usernames, user contact information, user demographics, and the like. In examples, user data 412 may include data that a smart device may have generated. For example, user data 412 may include the usage of a smart device, such as the frequency with which features are used, the duration of those features are used, and the like.

User data 412 may include feedback data. The feedback data may include feedback data associated with a medical product. A user of the medical product may have provided the feedback data. For example, the feedback data may indicate that a surgical stapler was easy to use or that a surgical stapler caused pain during use. The feedback data may be in the form of a review (e.g., a one to five-star rating with accompanying text). The feedback data may be in the form of survey responses. For example, a user may have been asked to rate their satisfaction with a surgical stapler on a scale of one to five, with five being the most satisfied.

The feedback data may be used by medical product assistant 402 to provide recommendations to users. For example, if a user is searching for a surgical stapler, medical product assistant 402 may use the feedback data to recommend a surgical stapler that has received high user ratings.

User data 412 may include preference data. The preference data may include preference data associated with a smart device user. The preference data may indicate the preferences of the user. For example, the preference data may indicate that the user prefers surgical staplers that are easy to use. The preference data may be in the form of survey responses. For example, a user may have been asked to rate the importance of various factors when choosing a surgical stapler (e.g., ease of use, price, brand).

Medical device data 414 may be accessed by medical product assistant 402. Medical device data 414 may include data from a medical device, such as an insulin pump. Medical device data 414 may include data about the medical device, such as the name of the manufacturer of the medical device, the type of medical device, the model number of the medical device, and the like. Medical device data 414 may include data captured by a medical device. For example, medical device data 414 may include sensor data captured by an insulin pump, a heart monitor, a blood pressure monitor, and the like. Medical device data 414 may include data that may indicate the usage of a medical device, such as data regarding the firing of a surgical stapler, the deployment of a stent, and the like.

Signal recognition 416 may be accessed by medical product assistant 402. Signal recognition 416 may be used to determine the identity of a medical device based on a signal emitted from the medical device, and/or a message that may have been sent by the medical device. For example, signal recognition 416 may identify a medical device based on how the medical device uses electricity, battery power, a wireless connection, a combination thereof, and the like. Signal recognition 416 may identify a device as a counterfeit based on a signal emitted from the device.

Signal recognition 416 may be able to determine that the power cycle used by the device indicates that the device is not authentic. For example, if the power cycle emitted by a device being checked does not match the power cycle of an authentic device, signal recognition 416 may determine that the device is a counterfeit.

Signal analysis AI model 420 may be accessed by signal recognition 416. Signal analysis AI model 420 may include one or more AI models that may be used to identify a signal associated with a known medical product. For example, signal analysis AI model 420 may include an AI model that may be used to identify a signal emitted and/or used by a surgical stapler, as such power cycles used by a motor of the surgical stapler. Signal analysis AI model 420 may include a model that may have been trained on data from device signal data 424.

Device signal data 424 may be accessed by signal analysis AI model 420. Device signal data 424 may include signal data that may have been captured and/or produced by a medical device. The signal data may include power cycles, battery life, wireless connection data, and the like. The signal data may be captured by a sensor associated with a medical device, such as a hall effect sensor. The signal data may be captured by a camera, such as an infrared camera. A microphone may capture the signal data.

Signal analysis AI model 420 may be accessed by signal recognition 416. Signal analysis AI model 420 may include one or more AI models that may be used to identify a signal associated with a known medical product. For example, signal analysis AI model 420 may include an AI model that may be used to identify a signal emitted and/or used by a surgical stapler, such as power cycles used by a motor of the surgical stapler. Signal analysis AI model 420 may include a model that may have been trained on data from device signal data 424.

Device signal data 424 may be accessed by signal analysis AI model 420. Device signal data 424 may include signal data that may have been captured and/or produced by a medical device. The signal data may include power cycles, battery life, wireless connection data, and the like. The signal data may be captured by a sensor associated with a medical device, such as a hall effect sensor. The signal data may be captured by a camera, such as an infrared camera. A microphone may capture the signal data.

Image recognition 418 may be accessed by medical product assistant 402. Image recognition 418 may be used to determine the identity of a medical device based on an image of the medical device. For example, image recognition 418 may be able to identify a surgical stapler based on an image of the surgical stapler. Image recognition 418 may be used to determine whether a particular feature is present in an image. For example, image recognition 418 may be used to determine whether a surgical stapler is missing a safety feature by looking for the presence or absence of the safety feature in an image of the surgical stapler.

Image recognition 418 may be used to determine the identity of a medical device based on a video of the medical device. For example, image recognition 418 may be used to identify a surgical stapler based on a video of the surgical stapler in use. Image recognition 418 may determine whether a particular feature may be present in a video. For example, Image recognition 418 may be used to determine from a portion of an object that the object may be a medical product.

Image recognition 418 may determine if a device is a counterfeit device using an image and/or video of the device. For example, image recognition 418 may determine that a surgical stapler is counterfeit based on the size, shape, and/or color of the surgical stapler.

Image recognition 418 may be and/or may provide the functionality of medical product recognition module 316, medical package recognition module 318, and/or counterfeit detection module 320 as shown in FIG. 3.

Referring to FIG. 4, diagnosis AI model 426 may be accessed by image recognition 418. Diagnosis AI model 426 may include one or more elements that may be used to diagnose a medical condition associated with a patient. The medical condition may have been treated with a medical product or may have been caused by the medical product. For example, the medical condition may be a healing wound caused by an incision during a medical procedure.

Diagnosis AI model 426 may be used with an image of a patient to diagnose a medical condition that the patient is experiencing. For example, a patient may send a picture of their healing wound to medical product assistant 402, which may be received by diagnosis AI model 426. The diagnosis AI model 426 analyzes images and may determine if the wound is infected.

Medical condition data 428 may be accessed by diagnosis AI model 426. For example, diagnosis AI model 426 may have one or more data sets from medical condition data 428. The data sets may include images of infected and non-infected wounds: images of cancerous and non-cancerous moles, images of healthy and unhealthy tissue, and the like.

Diagnosis AI model 426 may access medical condition data 428 to provide a diagnosis. Medical condition data 428 may include information used to diagnose a medical condition. For example, medical condition data 428 may include a list of symptoms associated with an infection, biomarkers associated with an infection, and the like. Medical condition data 428 may include information associated with a treatment for a medical condition. For example, if diagnosis AI model 426 determines that a wound is infected, it may recommend an antibiotic ointment to treat the infection and/or generate a treatment plan for the medical condition.

Product detection AI model 430 may be accessed by image recognition 418. Product detection AI model 430 may include one or more AI models that may be used to identify a medical product and/or a package associated with a medical product. For example, the medical product may be a medical device, such as a pacemaker, which may be implanted in a patient during a medical procedure. During a surgical procedure, a user may take a video of the pacemaker. Image recognition 418 may send the video to product detection AI model 430, which may analyze the video and may identify the medical product in the video as a pacemaker.

Counterfeit detection AI model 432 may be accessed by image recognition 418. Counterfeit detection AI model 432 may include one or more AI models that may be used to determine if a medical product is counterfeit. For example, a medical product may be a surgical stapler that a hospital purchased from an online retailer. A user may take a photo of a package that includes the surgical stapler and send the image-to-image recognition 418. Image recognition 418 may send the image to counterfeit detection AI model 432. Counterfeit detection AI model 432 may analyze the image and/or the package and determine that the surgical stapler is counterfeit.

Product detection data 434 may be accessed by product detection AI model 430 and/or counterfeit detection AI model 432. Product detection data 434 may include information associated with a medical product. For example, product detection data 434 may include a list of identifying features of the medical product, a list of authorized retailers for the medical product, images of the medical product, computer-aided drawings associated with the medical product, images of packaging associated with the medical product, computer-aided drawings of packaging associated with the medical product, blueprints of the medical product, blueprints of packaging associated with the medical product, and the like.

Synthetic product detection data 436 may be accessed by product detection AI model 430 and/or counterfeit detection AI model 432. Synthetic product detection data 436 may include synthetic data associated with one or more medical products, medical product packages, counterfeit medical products, and/or counterfeit medical packages. The synthetic data may include data, such as images, which were generated by synthetic data generator 440.

Synthetic data may be algorithmically generated data that imitates real data and may substitute for datasets used for modeling and training in artificial intelligence. The synthetic data generator 440 may generate one or more synthetic images of a medical product, a medical product package, a counterfeit medical product, and/or a counterfeit medical product package from image data, video data, and/or from computer automated drawings (CAD models). As described herein, the synthetic data (e.g., images) may be used to train a machine learning model to recognize the medical product in the image and/or video data. In examples, features learned or extracted from synthetic data may be transferred to real data to improve the performance of a machine learning model on the real data. For example, a machine learning model may be trained using synthetic images of medical devices that include known good and bad examples of the medical devices (e.g., genuine vs. counterfeit). The machine learning model may then be applied to real images to predict whether the medical devices in the real images are genuine or counterfeit.

Synthetic data may include images designed to force an artificial intelligence to focus on a product's shape and/or aspect, excluding an image background. For example, synthetic data generator 440 may generate an image by imposing an image of a medical product, a medical product package, and/or a counterfeit medical product on a generated background. The background may include a medical setting, such as an operating room, a hospital ward, a doctor's office, and the like. The background may include items found within a medical setting, such as medical equipment, surgical tools, and the like.

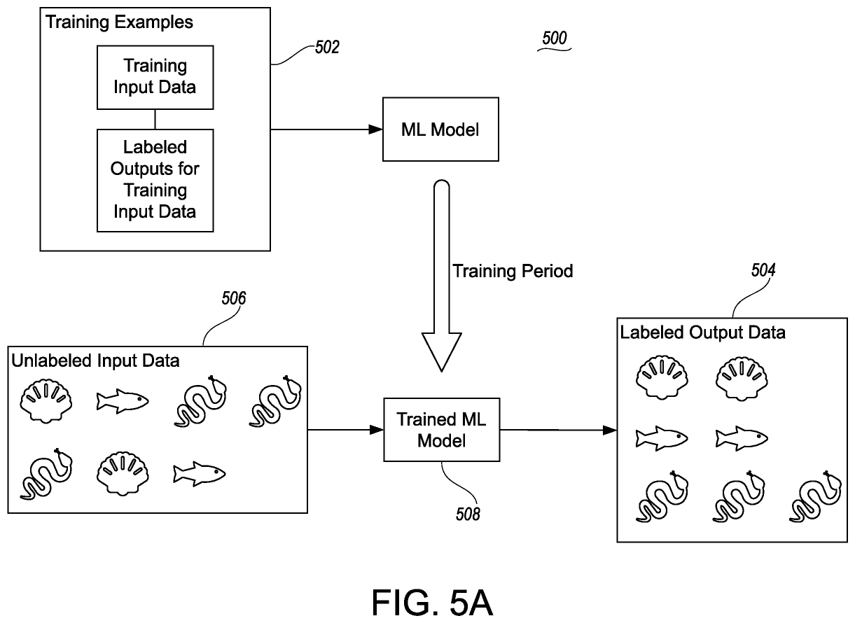

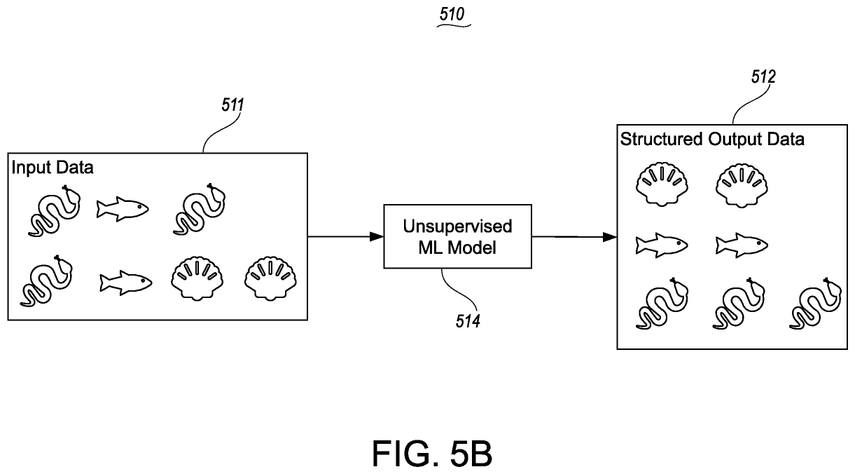

FIG. 5A illustrates an example of a supervised learning framework 500. The training data (e.g., training examples 502, for example, as shown in FIG. 5) may consist of a set of training examples (e.g., input data mapped to labeled outputs, for example, as shown in FIG. 5). A training example 502 may include one or more inputs and one or more labeled outputs. The labeled output(s) may serve as supervisory feedback. A training example 502 may be represented by an array or vector in a mathematical model, sometimes called a feature vector. The training data may be represented by row(s) of feature vectors, constituting a matrix. Through iterative optimization of an objective function (e.g., cost function), a supervised learning algorithm may learn a function (e.g., a prediction function) that may be used to predict the output associated with one or more new inputs. A suitably trained prediction function (e.g., trained ML model 508) may determine the output (e.g., labeled output data 504) for one or more inputs (e.g., unlabeled input data 506) that may not have been a part of the training data (e.g., input data without mapped labeled outputs, for example, as shown in FIG. 5A). Example algorithms may include linear regression, logistic regression, neural network, nearest neighbor, Naive Bayes, decision trees, SVM, and the like. Example problems solvable by supervised learning algorithms may include classification, regression problems, and the like.

Machine learning may be unsupervised (e.g., unsupervised learning). FIG. 5B illustrates an example of an unsupervised learning framework 510. An unsupervised learning algorithm (e.g., unsupervised ML model 514) may train on a dataset containing inputs (e.g., input data 511) and find a structure (e.g., structured output data 512) in the data. The structure may be found, for example, using pattern detection and/or descriptive modeling. The structure (e.g., structured output data 512) in the data may be similar to a grouping or clustering of data points. As such, algorithm (e.g., unsupervised ML model 514 may learn from training data that may not have been labeled. Instead of responding to supervisory feedback, an unsupervised learning algorithm may identify commonalities in training data and react based on the presence or absence of such commonalities in each training datum. For example, the training may include operating on training input data to generate a model and/or output with particular energy (e.g., such as a cost function), where such energy may be used to refine the model further (e.g., to define a model that minimizes the cost function given the training input data). Example algorithms may include the Apriori algorithm, K-Means, K-Nearest Neighbors (KNN), K-Medians, and the like. Example problems solvable by unsupervised learning algorithms may include clustering problems, anomaly/outlier detection problems, and the like.

Machine learning may be semi-supervised (e.g., semi-supervised learning). A semi-supervised learning algorithm may be used in scenarios where the cost to label data is high (e.g., because it requires skilled experts to label the data) and there are limited labels for the data. Semi-supervised learning models may exploit the idea that although group memberships of unlabeled data are unknown, the data still carries important information about the group parameters.

Machine learning may include reinforcement learning, an area of machine learning concerned with how software agents may take actions in an environment to maximize a notion of cumulative reward. Reinforcement learning algorithms may not assume knowledge of an exact mathematical model of the environment (e.g., represented by a Markov decision process (MDP)) and may be used when exact models are not feasible. Reinforcement learning algorithms may be used in autonomous vehicles or in learning to play a game against a human opponent. Examples of algorithms may include Q-Learning, Temporal Difference (TD), Deep Adversarial Networks, and the like.

Reinforcement learning may include an algorithm (e.g., agent) continuously iteratively learning from the environment. In the training process, the agent may learn from experiences of the environment until the agent explores the full range of states (e.g., possible states). A type of problem may define reinforcement learning. Solutions of reinforcement learning may be classed as reinforcement learning algorithms. In a problem, an agent may select an action (e.g., the best action) based on the agent's current state. If a process is repeated, the problem may be referred to as an MDP.

For example, reinforcement learning may include operational processes. An operation process in reinforcement learning may include the agent observing an input state. An operation process in reinforcement learning may include using a decision-making function to make the agent act. An operation process may include (e.g., after an action may be performed) the agent receiving a reward and/or reinforcement from the environment. An operation process in reinforcement learning may include storing the state-action pair information about the reward.

Machine learning may be a part of a technology platform called cognitive computing (CC), which may comprise various disciplines such as computer science and cognitive science. CC systems may be capable of learning at scale, reasoning with purpose, and interacting with humans naturally. Using self-teaching algorithms that may use data mining, visual recognition, and/or natural language processing, a CC system may be capable of solving problems and optimizing human processes.

The output of machine learning's training process may be a model for predicting outcome(s) on a new dataset. For example, a linear regression learning algorithm may be a cost function that may minimize the prediction errors of a linear prediction function during the training process by adjusting the coefficients and constants of the linear prediction function. When a minimum may be reached, the linear prediction function with adjusted coefficients may be deemed trained and constitutes the model the training process has produced. For example, a neural network (NN) algorithm (e.g., multilayer perceptrons (MLP)) for classification may include a hypothesis function represented by a network of layers of nodes that are assigned with biases and interconnected with weight connections. The hypothesis function may be a non-linear function (e.g., a highly non-linear function) that may include linear functions and logistic functions nested together with the outermost layer consisting of one or more logistic functions. The NN algorithm may include a cost function to minimize classification errors by adjusting the biases and weights through feedforward propagation and backward propagation. When a global minimum may be reached, the optimized hypothesis function with its layers of adjusted biases and weights may be deemed trained and constitute the model the training process has produced.

Data collection may be performed for machine learning as a first stage of the machine learning lifecycle. Data collection may include processes such as identifying various sources, collecting data from the sources, integrating the data, and the like. For example, for training a machine learning model for predicting surgical complications and/or post-surgical recovery rates, data sources containing pre-surgical data, such as a patient's medical conditions and biomarker measurement data, may be identified. Such data sources may be a patient's electronic medical records (EMR), a computing system storing the patient's pre-surgical biomarker measurement data, and/or other like data stores. The data from such data sources may be retrieved and stored in a central location for further processing in the machine learning lifecycle. The data from such sources may be linked (e.g., logically linked) and accessed as if they were centrally stored. Surgical data and/or post-surgical data may be similarly identified and collected.

Further, the collected data may be integrated. For example, a patient's pre-surgical medical record data, pre-surgical biomarker measurement data, pre-surgical data, surgical data, and/or post-surgical data may be combined into a record for the patient. The record for the patient may be an EMR.

Data preparation may be performed for machine learning as another stage of the machine learning lifecycle. Data preparation may include data preprocessing steps such as data formatting, cleaning, and sampling. For example, the collected data may not be in a data format suitable for training a model. Such data may be converted to a flat-file format for model training. Such data may be mapped to numeric values for model training. Such identifying data may be removed before model training. For example, identifying data may be removed for privacy reasons. As another example, data may be removed because more data may be available than used for model training. A subset of the available data may be randomly sampled and selected for model training, and the remainder may be discarded.

Data preparation may include data transforming procedures (e.g., after preprocessing), such as scaling and aggregation. For example, preprocessed data may include data values in a mixture of scales. For example, these values may be scaled up or down, between 0 and 1 for model training. For example, preprocessed data may include values that carry more meaning when aggregated.

Model training may be another aspect of the machine learning lifecycle. The model training process described herein may depend on the machine learning algorithm used. A model may be deemed suitably trained after it has been trained, cross-validated, and tested. Accordingly, the dataset from the data preparation stage (e.g., an input dataset) may be divided into a training dataset (e.g., 60% of the input dataset), a validation dataset (e.g., 20% of the input dataset), and a test dataset (e.g., 20% of the input dataset). After the model has been trained on the training dataset, the model may be run against the validation dataset to reduce overfitting. If the model's accuracy were to decrease when run against the validation dataset when the model's accuracy has been increasing, this may indicate a problem of overfitting. The test dataset may be used to test the accuracy of the final model to determine whether it is ready for deployment or whether more training may be requested (e.g., required).

Model deployment may be another aspect of the machine learning lifecycle. The model may be deployed as a part of a standalone computer program. The model may be deployed as a part of a larger computing system. A model may be deployed with model performance parameters(s). Performance parameters may monitor the model accuracy as it is used for predicting a production dataset. For example, such parameters may keep track of false positives and false positives for a classification model. Such parameters may store the false positives and positives for further processing to improve the model's accuracy.

Post-deployment model updates may be another aspect of the machine learning cycle. For example, a deployed model may be updated as false positives and/or false positives are predicted on production data. In an example, for a deployed MLP model for classification, as false positives occur, the deployed MLP model may be updated to increase the probable cutoff for predicting a positive to reduce false positives. In an example, for a deployed MLP model for classification, as false negatives occur, the deployed MLP model may be updated to decrease the probable cutoff for predicting a positive to reduce false negatives. In an example, for a deployed MLP model for the classification of surgical complications, as both false positives and false negatives occur, the deployed MLP model may be updated to decrease the probably cutoff for predicting a positive to reduce false negatives because it may be less critical to predicting a false positive than a false negative.

For example, a deployed model may be updated as more live production data becomes available as training data. In such cases, the deployed model may be further trained, validated, and tested with additional live production data. In an example, the updated biases and weights of a further-trained MLP model may update the deployed MLP model's biases and weights. Those skilled in the art recognize that post-deployment model updates may not be a one-time occurrence and may occur as frequently as suitable for improving the deployed model's accuracy.

ML techniques may be used independently of each other or in combination. Different problems and/or datasets may benefit from using different ML techniques (e.g., combinations of ML techniques). Different training types for models may be better suited for a certain problem and/or dataset. An optimal algorithm (e.g., a combination of ML techniques) and/or training type may be determined for a specific usage, problem, and/or dataset. For example, a process may be performed for one or more of the following: choose a data reduction type, choose a configuration for a model and/or algorithm, determine a location for the data reduction, choose an efficiency of the reduction and/or result, and the like.

For example, an ML technique, or a combination of ML techniques, may be determined for a particular problem and/or use case. Multiple data reduction and/or data analysis processes may be performed to determine accuracy, efficiency, and/or compatibility associated with a dataset. For example, a first ML technique (e.g., the first set of combined ML techniques) may be used on a dataset to perform data reduction and/or data analysis. The first ML technique may produce a first output. A second ML technique (e.g., a second set of combined ML techniques) may be used on the dataset (e.g., the same dataset) to perform data reduction and/or data analysis. The second ML technique may produce a second output. The first output may be compared with the second output to determine which ML technique produced more desirable results (e.g., more efficient and accurate results). Multiple ML techniques may be compared with the same dataset to determine the optimal ML technique(s) to use on a future similar dataset and/or problem.

In examples, in a medical context, a surgeon or healthcare professional may give feedback to ML techniques and/or models used on a dataset. The surgeon may input feedback to the weighted results of an ML model. The model may use the feedback as input to determine a reduction method for future analyses.

A data analysis method (e.g., ML techniques to be used in the data analysis method) may be determined based on the dataset. For example, the origin of the data may influence the type of data analysis method to be used on the dataset. System resources available may be used to determine the data analysis method for a given dataset. The data magnitude, for example, may be considered in determining a data analysis method. For example, the need for datasets exterior to the local processing level or magnitude of operational responses may be considered. For example, small device changes may be made with local data; major device operation changes may request (e.g., require) global compilation and verification.

ML techniques may be applied to medical product information (e.g., a combination of information flows of medical product information) to generate ML models.

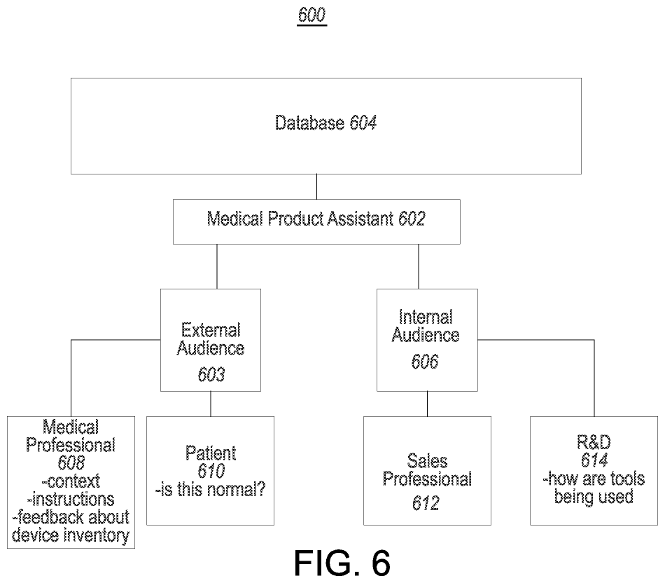

FIG. 6 depicts a block diagram of a system for providing a medical product assistant that may be able to respond to requests from one or more audiences and/or contexts. System 600 may include medical product assistant 602 and/or database 604. Database 604 may include any of the data described. For example, database 604 may include medical product data, marketing data, user data, medical data, artificial intelligence models, a combination thereof, and the like. Medical product assistant 602 may be or may be able to provide the functionality provided by medical product assistant module 314, shown in FIG. 3, and/or medical product assistant 402, shown in FIG. 4.

Referring again to FIG. 6, medical product assistant 602 may be able to respond to inquiries from internal audiences and external audiences. Medical product assistant 602 may distinguish audiences based on whether an audience is internal to an entity, such as a manufacturer, or external to the entity. For example, medical product assistant 602 may be used by a manufacturer, and an internal audience may include sales professionals, research and development, and other employees of the manufacturer. An example, medical product assistant 602 may be used by a manufacturer, and an external audience may include medical professionals and/or patients.

External audience 603 may be external to an entity hosting and/or deploying medical product assistant 602. External audience 603 may include medical professional 608 and/or patient 610.

Internal audience 606 may be internal to an entity that is hosting and/or deploying medical product assistant 602. Internal audience 606 may include sales professionals 612 and/or research and development 614.

Medical product assistant 602 may be able to provide content for a variety of use cases. For example, medical product assistant 602 may be used by a sales professional (e.g., sales professional 612) during a sales call to quickly look up information about products, such as product information, and/or to generate product-specific content. In example, medical product assistant 602 may be used by research and development professionals (e.g., R&D 614) during the product development process to quickly look up information about products, such as regulatory data, and/or to generate clinical trial-specific content.

Medical product assistant 602 may be used by a medical professional (e.g., medical professional 608) to quickly look up information about products, such as product information and/or regulatory data. Medical product assistant 602 may also be used by medical professional 608 to provide patient-specific content. For example, a doctor may use medical product assistant 602 to determine the content that may be provided to a patient before a procedure. The content may include information about the procedure, risks, and benefits of the procedure, and/or post-operative care instructions.

Medical product assistant 602 may also be used by patent 610 to determine patient-specific content 624. For example, medical product assistant 602 may select the content that may be provided to patient 610 before a procedure. The content may include information about the procedure, risks, and benefits of the procedure, and/or post-operative care instructions.

Medical product assistant 602 may be used to generate marketing materials. For example, medical product assistant 602 may synthesize data, analyze market trends, and utilize AI-driven algorithms to create marketing content. This may include brochures, product descriptions, promotional videos, and advertisements that may be designed to reach and appeal to the target audience. These marketing materials may be personalized to meet the preferences of potential users, which may enhance the user engagement.

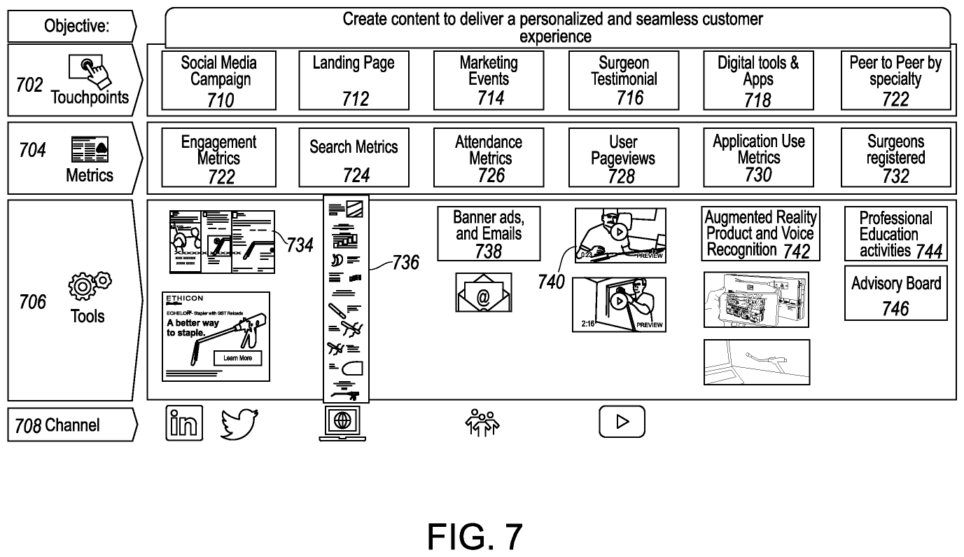

FIG. 7 depicts a block diagram of a system for providing a medical product assistant for delivering a personalized customer experience. A medical product assistant may use one or more touch points, metrics, tools, and/or channels to provide a customized experience for a user. At 702, touchpoints may be a social media campaign (e.g., social media campaign 710), a landing page (e.g., landing page 712), an event (e.g., marketing event 714), a testimonial event (e.g., surgeon testimonial 716), a digital tool, a digital application (e.g., digital tools and applications), a peer recommendation (e.g., peer recommendation 720), a combination thereof, and the like. At 704, a metric may be an engagement rate (e.g., engagement metrics 722), a lead rate, a search volume, a search term (e.g., search metrics 724), event attendance (e.g., attendance metrics 726), a user page views (e.g., user pageviews 728), a number of active users (e.g., user pageviews 728), a time-in-tool metric (e.g., application use metrics 730), a number of users registered (e.g., surgeons registered 732), a combination thereof, and the like. At 706, a tool may be a webpage (e.g., webpage 736), a banner ad (e.g., banner ads and emails 738), an email (e.g., banner ads and emails 738), a social media post (e.g., social media post 734), a video (e.g., video 740), an augmented reality product (e.g., augmented reality product and voice recognition 742), a voice recognition product (e.g., augmented reality product and voice recognition 742), a professional education activity (e.g., professional education activity 744), and advisory board (e.g., advisory board 746), a webinar, a combination thereof, and the like. At 708, a channel may be a social media channel such as LinkedIn, Twitter, and the like. The channel may be a web page, a YouTube destination, or a user.

At 702, one or more touchpoints may be provided by the medical product assistant to provide a personalized user experience. The medical product assistant may generate and manage personalized social media campaigns, such as social media campaign 710. For example, by analyzing user interactions, preferences, and behaviors, the medical product assistant may tailor content to create an enhanced and/or individualized user experience. The medical product assistant may integrate with various social media platforms, such as one or more channels at 708, for example, to ensure consistent and/or effective dissemination of medical product information, which may increase engagement and interaction with potential users. The medical product assistant may optimize social media campaign 710 by monitoring metrics, such as engagement metrics 722, which may include engagement rates and/or spend leads. The medical product assistant may manage social media campaign 710 to create social media post 734, which may be published via a social media channel at 708.

The medical product assistant may create, manage, and/or optimize landing pages, such as landing page 712. For example, the medical product assistant may assess user behavior and preferences to present information user-friendly and engagingly via landing page 712. The medical product assistant may configure landing page 712 to create and/or modify webpage 736, which may highlight the benefits and features of a medical product. Webpage 736 may be designed to assist in addressing the requests and concerns of the visitors. The medical product assistant may integrate webpage 736 with one or more channels at 708. The medical product assistant may optimize landing page 712 by monitoring metrics, such as search metrics 724, which may include search volume, search terms, conversion rates, and the like. Search metrics 724 may be used to create and/or modify webpage 736.

The medical product assistant may create, manage, and/or optimize marketing events, such as marketing event 714. For example, the medical product assistant may organize and manage marketing event 714 to promote a medical product. The medical product assistant may analyze metrics, such as attendance metrics 726, to optimize an event (e.g., event content), to tailor the event to the interests and needs of the participants. The medical product assistant may create an email, such as banner ads and emails 738, to invite a user to marketing event 714. The medical product assistant may customize the email for the user.

The medical product assistant may create, manage, and/or optimize user testimonials, such as surgeon testimonial 716. For example, the medical product assistant may identify testimonials, such as video 740, from surgeons with experience using a medical product. The medical product assistant may use metrics, such as user pageviews 728, to analyze and present the most relevant and impactful testimonials. By providing real-world experiences and opinions from respected medical professionals, the medical product assistant may enhance the reputation of the medical product and may aid in building a trustworthy relationship with potential users.

The medical product assistant may create, manage, and/or optimize applications, such as digital tools and applications 718. The medical product assistant may provide a suite of digital solutions designed to augment the user experience with the medical product. The medical product assistant uses metrics, such as application use metrics 730, to assist in the development and optimization of tools and applications. For example, the metrics may be used to assist in making an application, such as augmented reality product and voice recognition 742, personalized and user-friendly. The medical product assistant may customize augmented reality product and voice recognition 742 to provide a digital solution that may enrich the interaction with the medical product, may offer additional resources, support, and information, and may contribute to an improved user experience.

The medical product assistant may create, manage, and/or optimize peer recommendations, such as peer recommendation 720. For example, the medical product assistant may use peer recommendation 720 to facilitate discussions between medical professionals. By analyzing the specialties and interests of the medical professionals using metrics, such as surgeons registered 732 (e.g., surgeons registered for a professional education event), the medical product assistant may tailor discussion content, ensuring relevance and engagement. The medical product assistant may use metrics to recommend peer recommendation 720 to a user. The medical product assistant may use metrics to organize professional education activity 744 and/or advisory board 746. For example, the medical product assistant may determine that the number of surgeons registered at an education event satisfied a criterion based on the metrics and may recommend the creation of another event to an advisory board. The medical product assistant may provide a social media campaign, such as social media campaign 710. A personalized experience provided by the social media campaign may be measured using engagement rates, such as engagement metrics 722, for the social media post, such as social media post 734, and/or one or more web pages. The social media campaign may utilize one or more channels at 706, such as Twitter.

The medical product assistant may provide a webpage, such as landing page 712 and/or webpage 736. A personalized experience for the webpage, such as webpage 736, may be measured using metrics, such as search metrics 724, which may include search volume and/or search terms.

The medical product assistant may provide content for an event, such as marketing event 74. The content may be provided based on metrics, such as attendance metrics 726. The content for the event may be provided via banner ads, emails, and the like. For example, the content may be provided by banner ads and emails 738. A personalized experience may be provided by customizing the content for a recipient of the content.

The medical product assistant may provide a surgeon testimonial, such as surgeon testimonial 716, based on an inquiry from a user. A personalized experience for the user provided by the surgeon testimonial may be measured using metrics, such as user pageviews 728, which may include user page views. The personalized experience for the user may include video 740.

The medical product assistant may provide a customized software application for a user, such as digital tools and applications 718. The personalized experience for the software application may be measured by using metrics, such as application use metrics 730. For example, the medical product assistant may determine the number of users that use the software, the amount of time the users use the software, and the like. The personalized experience may include augmented reality and/or voice recognition, such as augmented reality product and voice recognition 742.

The medical product assistant may provide content, such as peer recommendation 720, for a webinar, such as professional education activity 744, which may be intended for one or more medical professionals. The content for the event may be customized for medical professionals based on metrics, such as surgeons registered 732, to provide a personalized experience. The personalized experience may be measured by determining the number of medical professionals that may register for the event. The personalized experience may include recommendations made by an advisory board, such as advisory board 746.

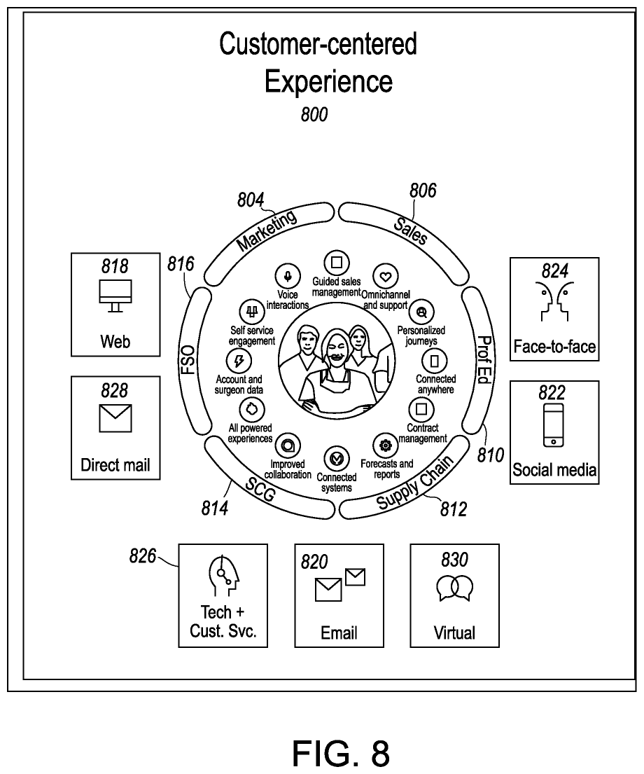

FIG. 8 depicts a block diagram of a system for providing a medical product assistant for delivering a personalized customer experience, such as customer-centered experience 800. At 802, the medical product assistant may use marketing material at 804, sales material at 806, FSO at 816, SSG at 814, supply chain data at 812, and/or professional educational data at 810 to provide a personalized customer experience. The medical product assistant may communicate with a user using direct mail at 828, the web at 818, email at 820, social media at 822, and the like. The medical practice system may provide content to one or more individuals who may interact face-to-face with a user at 824, or virtually at 830. At 826, the medical product assistant may provide content to an employee that may interact with a user, such as a customer service agent, a technical advisor, a salesperson, and the like.

The medical product assistant may customize voice interactions based on content that a user may be requesting. For example, the medical product assistant may provide guided sales management to a salesperson that may be requesting information regarding a medical product. The medical product assistant may provide omnichannel support such that the medical product assistant may provide support information for the medical product. The medical product assistant may provide a personal journey for a user based on data for a medical product. The medical product assistant may provide forecasts and/or reports regarding a medical product. The medical practice system may connect to one of our systems that may provide information regarding the medical product. The medical assistant may improve collaboration between one or more users. The medical assistant may utilize artificial intelligence to offer and/or identify information about a medical product. The medical product assistant may determine account data related to the medical product and/or surgical data associated with the medical product. The medical product assistant may assist in self-service engagement.

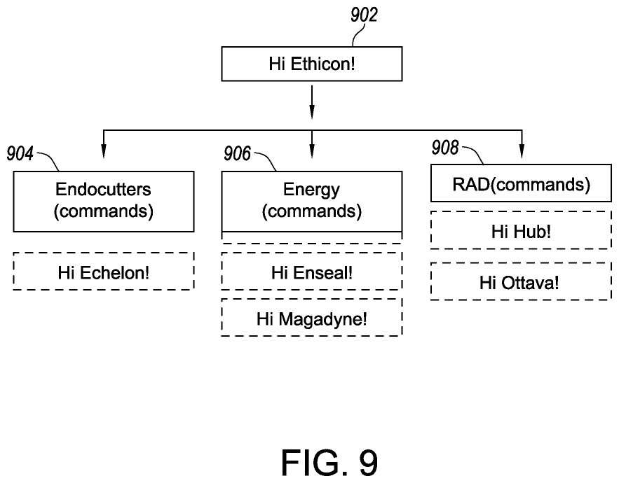

FIG. 9 depicts a block diagram that a medical product assistant may use for responding to one or more voice activation commands (e.g., wake words). In examples, the medical product assistant may be configured to respond to one or more voice activation commands at 902. A wake word may be a word or phrase a user may say to wake up an assistant and give it a command. The medical product assistant may include one or more wake words, such as “Hi Echelon” and/or “Hi Enseal.”

A medical product assistant may be able to determine that a wake work may be associated with a medical product. For example, the medical product assistant may determine that a first wake word is associated with a first medical device and a second wake word may be associated with a second medical device. The medical product assistant may provide a first set of functionalities based on the first awake word, such as the ability to control the surgical hub. The medical product assistant may provide a second set of functionalities based on the second wake word, such as an ability to determine data from a surgical stapler.

In examples, a user may provide one or more voice commands to the medical product assistant. The medical product assistant may determine a set of functionalities to offer, based on one or more voice commands. In examples, the medical product assistant may include a natural language processing engine configured to process one or more voice commands and determine the set of functionalities to provide.

In examples, the medical product assistant may provide a first set of functionalities based on receiving a voice command that includes a first keyword and provide a second set of functionalities based on receiving a voice command that includes a second keyword.

The medical product assistant may receive a wake word at 902. The medical product assistant may determine that the wake word is associated with product information and may provide the user with product information. At 904, the medical product assistant may determine that the wake word is associated with an endocutter (e.g., the wake word may be “Hi Echelon!”), and the medical product assistant may perform an action associated with the endocutter. At 906, the medical product assistant may determine that the wake word is associated with an electrosurgical generator (e.g., the wake word may be “Hi Enseal!” or “Hi Magadyne!”), and the medical product assistant may perform an action associated with the electrosurgical generator. At 908, the medical product assistant may determine that the wake word is associated with a surgical robot (e.g., the wake word may be “Hi Hub!” or “Hi Ottava”), and the medical product assistant may perform an action associated with the surgical robot.

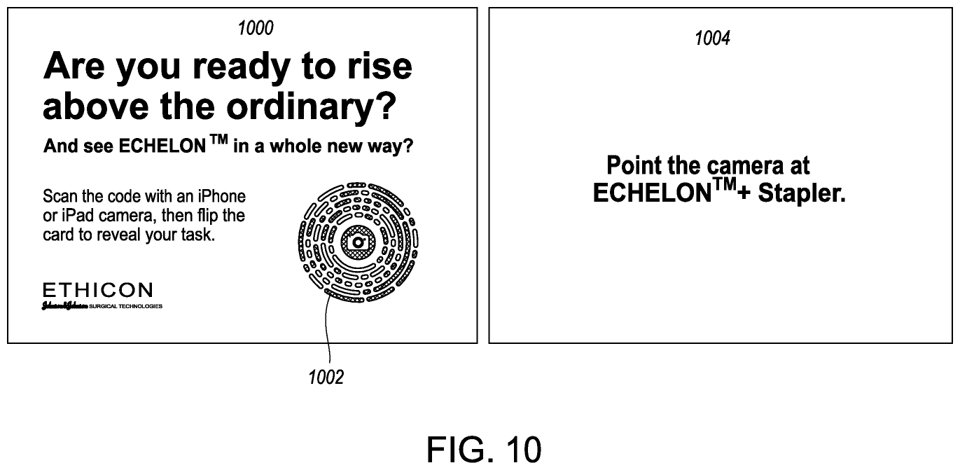

FIG. 10 is an example of a label and/or code that may be used to initiate a medical product assistant. The medical product assistant may be installed on a smart device, such as a smartphone. For example, a user may point a smartphone's camera at a label and/or code, such as the label 1000 and/or code 1002 shown in FIG. 10, which may cause the smartphone to initiate a download, installation, and/or start of the medical product assistant.

The label 1000 and/or code 1002 may trigger an app clip. An app clip may be a small part of an app that may be discoverable at the moment it may be requested. App clips may be fast and lightweight, so a user may quickly complete a task and then return to what they were doing. For example, the label and/or code shown in FIG. 10 may trigger an app clip, which may be a medical product assistant. As shown at 1004, the app clip may be loaded and may instruct the user to point a camera at a medical product, such as a surgical stapler.

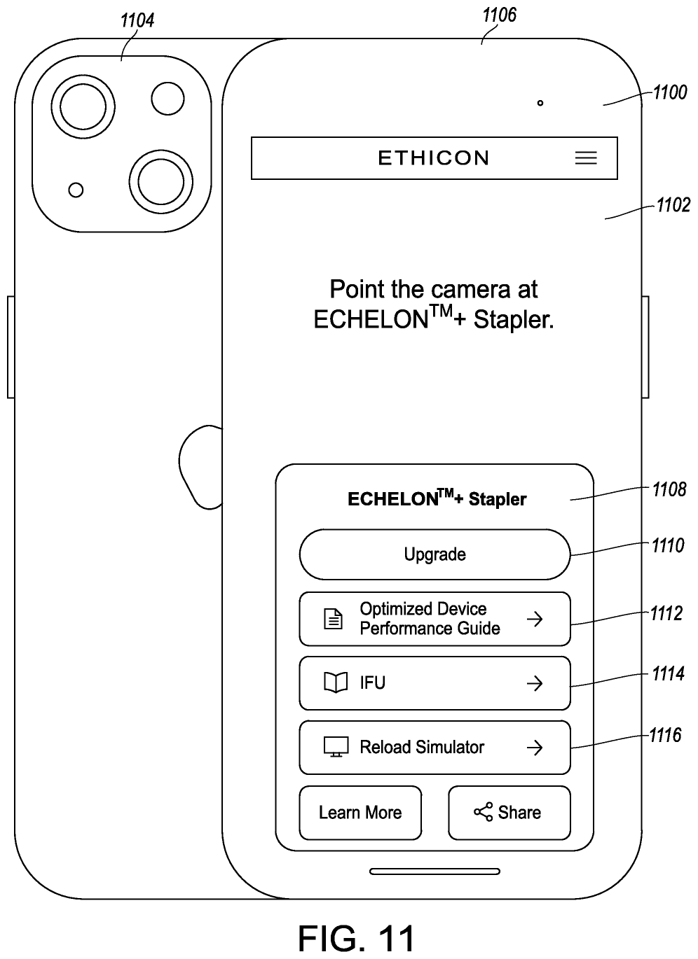

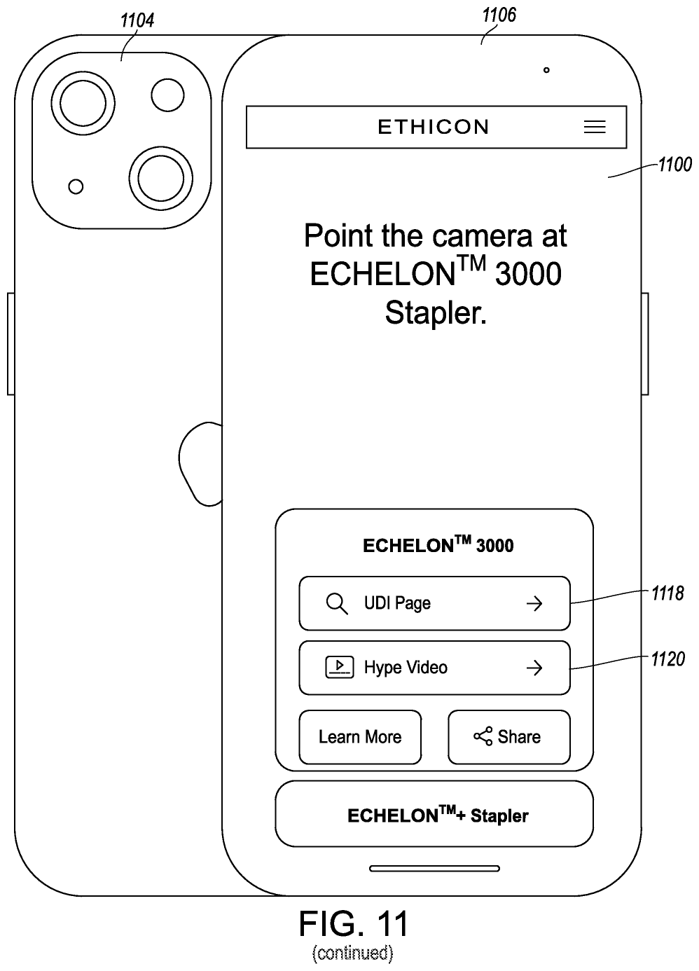

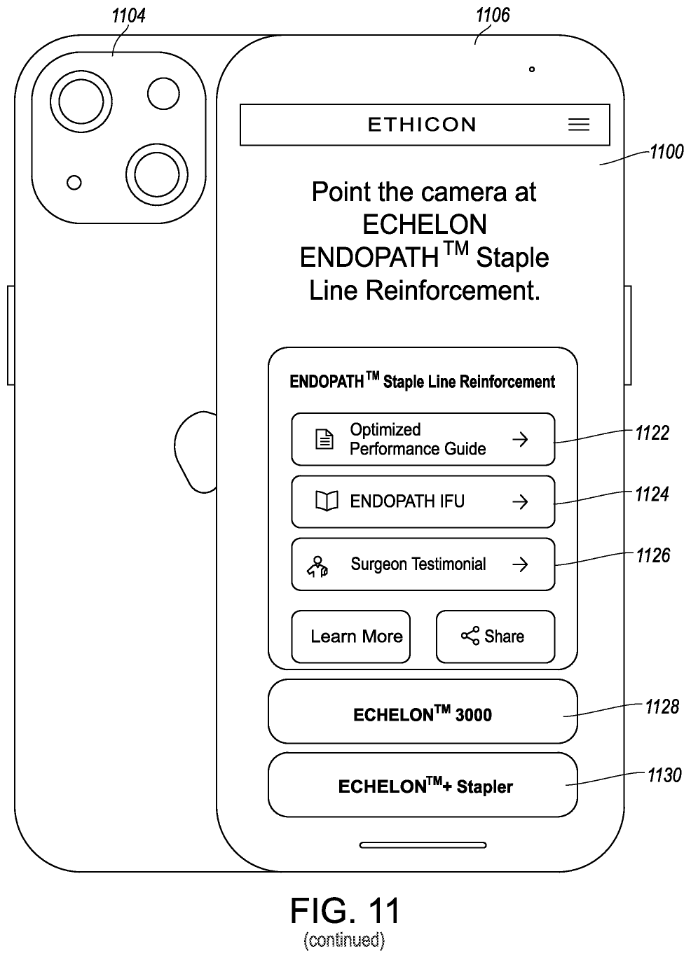

FIG. 11 depicts an example of one or more user interfaces that a medical product assistant may use to recognize, identify, and/or support a medical product. At 1102, the medical product assistant 1100 may request a user to point a camera, such as camera 1104, of smart phone 1106 at a medical product. The medical product may be a medical device, such as a stapler. The medical product may identify the medical product and may offer information to the user at 1108. The information may include instructions for use (IFU) at 1114 or 1124, a product guide at 1112 or 1122, a product simulator (such as a reload simulator for a surgical stapler) at 1116, a unique device identifier (UDI) at 1118, a webpage associated with a UDI, a webpage associated with the medical product, a video at 1120, a video with a user testimonial at 1126, marketing information, information regarding another product at 1128 or 1130, information regarding upgrading to another medical product and the like. For an example, at 1110, medical product assistant 1100 may offer to upgrade the software of the medical product, which may be an electronic surgical device.

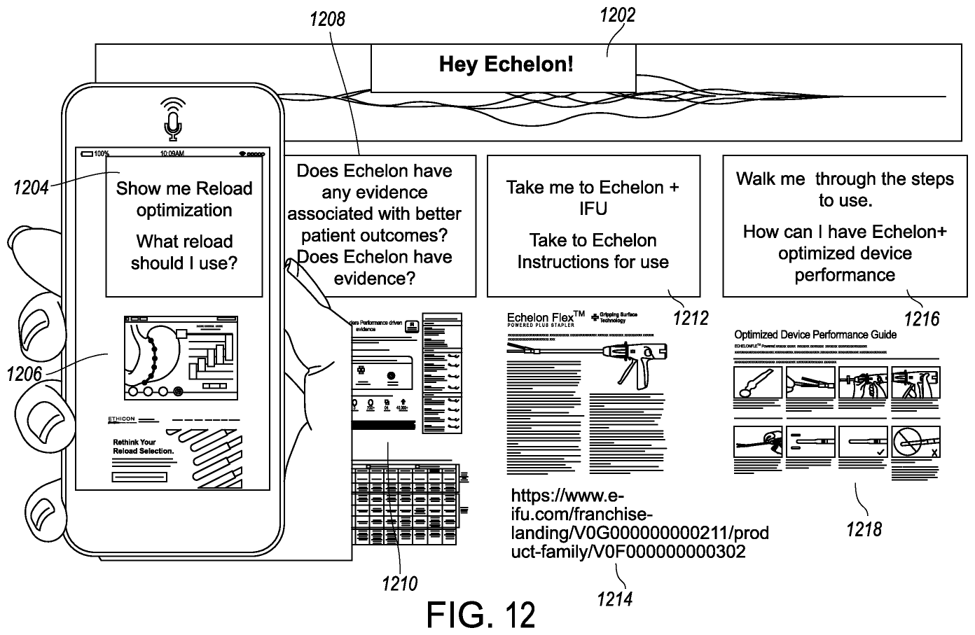

FIG. 12 depicts an example of one or more user interfaces that a medical product assistant may use to support a medical product. The medical product assistant may be triggered using a wake word at 1202. The medical product assistant may determine that the user has asked a question and may respond to the question. For example, at 1204 the user may ask a medical product assistant what surgical staples may be used for reloading a surgical stapler. At 1206, the medical product assistant may provide instructions, diagrams, and/or medical product information regarding a surgical staple/or a surgical stapler. For example, at 1208 a user may ask a medical product assistant if there is any evidence associated with a patient outcome and the medical, and at 1210, the medical product assistant may provide evidence and/or medical data associated with the medical product. At 1212, a medical product assistant may be asked to provide instructions for using a medical product, and at 1214, the medical product assistant may provide instructions for use (e.g., an IFU). For example, at 1216, the medical product assistant may be asked for instructions on how to use a medical product, and at 1218, the medical product assistant may provide a medical product guide.

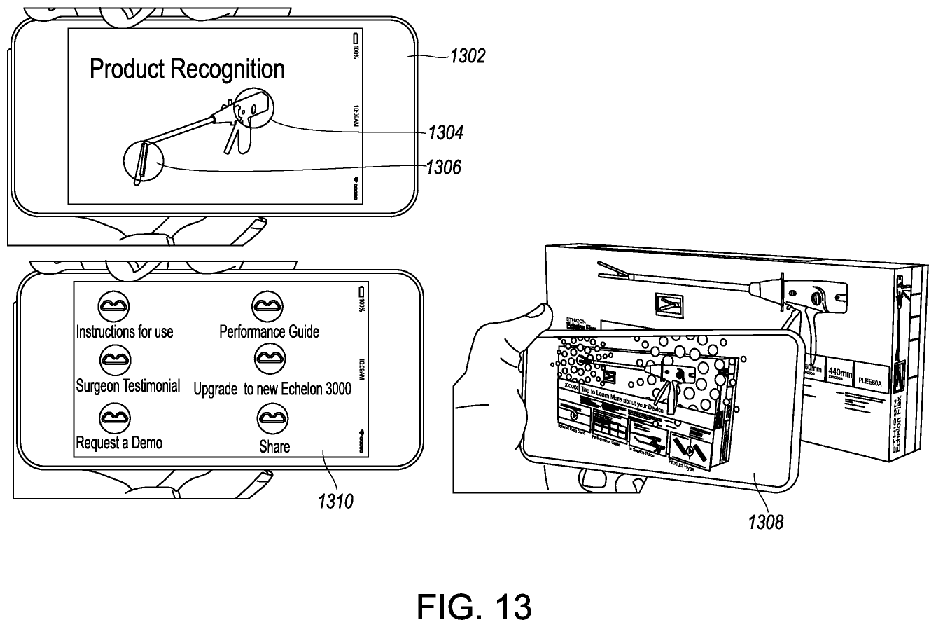

FIG. 13 depicts an example of one or more user interfaces that a medical product assistant may use for recognizing and/or identifying a medical product. At 1302, the medical product assistant may allow the user to point a smart device's camera toward a medical product. The medical product may be identified using a portion of the medical product, such as shown at 1304 and 1306. For example, the medical product assistant may identify a surgical stapler based on recognizing an anvil (e.g., 1306) and a handle (e.g., 1304) associated with the surgical stapler.

The medical product assistant may identify a medical product based on its color. For example, a medical product may be provided in a standardized color (e.g., white for a handle of a surgical stapler, green for a cartridge of surgical staples). The identification of standardized color may be used to identify a medical product. For example, the color may be a color that may be detected by an infrared sensor but may not be seen by the human eye.

The size of the medical product may be used to identify the medical product. The size of the medical product may be identified using one or more images of the medical product. The size of the medical product may be compared with sizes associated with known medical products to identify the medical product.

A combination of information about a medical product (e.g., shape, color, size, label, and/or image) may be used to determine the identity of the medical product. For example, a machine learning model may be trained to recognize medical products based on a combination of the medical product's shape, color, size, label, and/or image.

At 1308, the medical product assistant may be used to recognize a medical product package from an image and/or a video. A medical product assistant may be used to identify a box, bottle, blister pack, and the like that includes one or more doses of a medication product. The medical product assistant may be asked to determine an identifier for the medical product.

The shape of the medical product package may be used to identify the medical product. For example, a medical product may be recognized based on one or more edges in an image of the medical product package. One or more edges in the image of the medical product package may be identified using a machine-learning model as described herein.

The color of the medical product package may be used to identify the medical product. The color of the medical product package may be identified from an image of the medical product package. The color of the medical product package may be compared with colors associated with known medical product packages to identify the medical product.

The medical product assistant may identify a medical product package based on the color. For example, a medical product package may be provided in a standardized color (e.g., white for a box of the package, red for the text of the package, etc.). The identification of the medical product package with the standardized color may be used to identify the medical product. The color may be a color that may be detected by an infrared sensor but may not be seen by the human eye.

The size of the medical product package may be used to identify the medical product. The size of the medical product package may be identified using one or more images of the medical product package. The size of the medical product package may be compared with sizes associated with known medical product packages to identify the medical product.

A combination of information about the medical product package (e.g., shape, color, size, label, and/or image) may be used to determine the identity of the medical product. For example, a machine learning model may be trained to recognize medical product packages (e.g., and their contents) based on a combination of the shape, color, size, label, and/or image of the medical product package.

At 1310, the medical product assistant may provide information regarding an identified medical product. The information may include instructions for use, a surgeon testimonial, a demo, a performance guide, marketing material, an offer to upgrade the product, and the like.

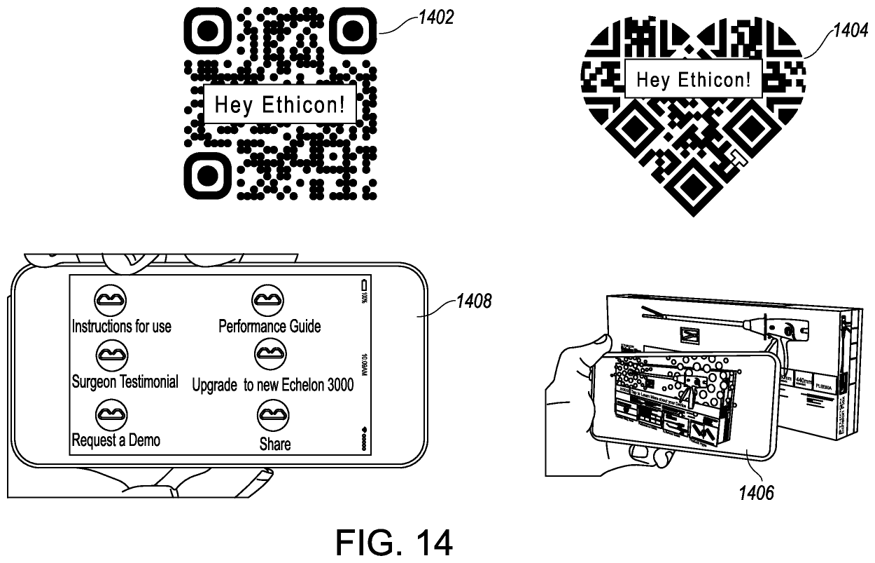

FIG. 14 depicts an example of one or more user interfaces that a medical product assistant may use for recognizing and/or identifying a medical product using encoded information associated with the medical product.

The medical product assistant may use text and/or images to identify a medical product. For example, a medical product and/or a medical product package may include identifying text. The medical product assistant may use OCR to process text to determine the medical product's identity and/or information about the medical product from the processed text

At 1406, the medical product assistant may identify a portion of a medical product and/or a medical product package within an image and may use that portion to determine the identity of the medical product. For example, the medical product assistant may identify a label on the medical product package. The label may include text and/or images that describe the medical product package. As shown in FIG. 14, the label may be a barcode, QR code, and the like. For example, the label may be QR code 1402 and/or QR code 1404. At 1408, the medical product assistant may identify and determine medical product information using the label.

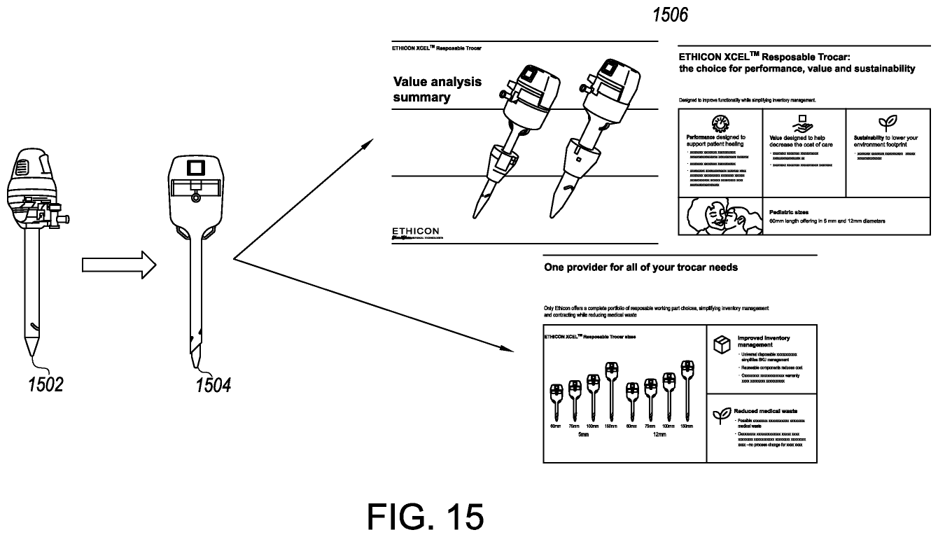

FIG. 15 depict an example of one or more user interfaces that may be used by a medical product assistant to provide marketing material associated with a medical product. At, 1502 the medical product assistant may identify a first medical product. The medical product assistant may determine that the first medical product is an older version of the medical product. At 1504, the medical product assistance may determine the second medical product that may be an approved version of the first medical product. The medical product assistant may determine product information for the first medical product and/or the second medical product. At 1506, the medical product assistant may determine marketing information that may indicate one or more reasons why the second medical product may be an improvement over the first medical product. The medical product assistant may present the marketing information, the first medical product information, and/or the second medical product information to a user.

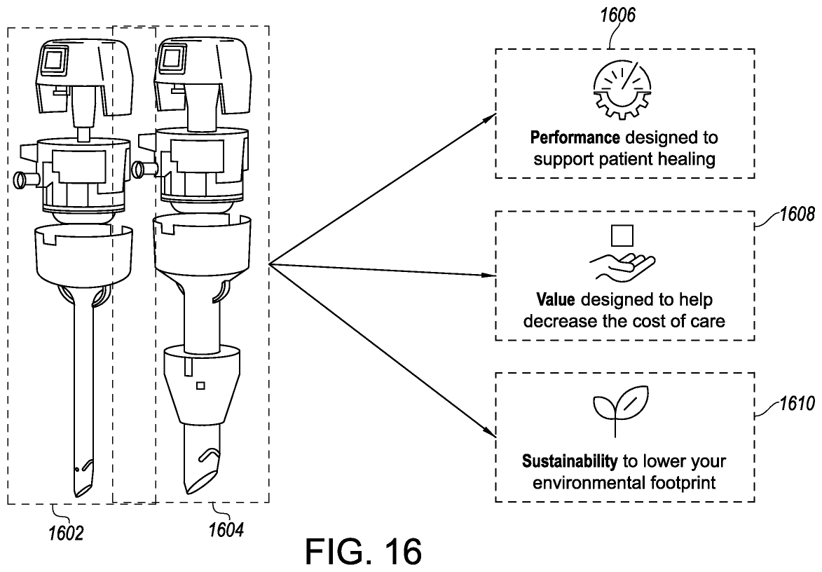

FIG. 16 depicts an example of one or more user interfaces that may be used by a medical product assistant to provide marketing material associated with a medical product. At, 1602 the medical product assistant may identify a first medical product. The medical product assistant may determine that the first medical product is an older version of the medical product. At 1604, the medical product assistance may determine the second medical product that may be an approved version of the first medical product. The medical product assistant may determine product information for the first medical product and/or the second medical product. The medical product assistant may determine marketing information that may indicate one or more reasons, such as at 1606, 1608, and 1610, why the second medical product may be an improvement over the first medical product. The medical product assistant may present the marketing information, the first medical product information, and/or the second medical product information to a user.

Various aspects of the subject matter described herein are set out in the following numbered examples:

Example 1—A device for providing a medical product assistant to identify a medical product, the device comprising a processor. The processor may be configured to receive a first message from a user interface wherein the first message indicates a request from a user to identify an object. The processor may be configured to determine a portion of the object using a camera of the device. The processor may be configured to generate a confidence level associated with a medical product, wherein the generated confidence level indicates a confidence that the object is the medical product, wherein the generated confidence level is based on the portion of the object and a product detection model, and wherein the product detection model has been trained using synthetic product detection data that comprises at least a computer generated image of the medical product. The processor may be configured to identify the object as the medical product when the confidence level satisfies a threshold. The processor may be configured to send a second message to the user interface when the object is identified, wherein the second message indicates that the object is the medical product, indicates a product identifier for the medical product, and indicates medical product data.

Example 2—The device in any of Example 1, wherein the processor is further configured to determine the portion of the object using the camera of the device by determining a video stream using the camera, determining an image from the video stream, and determining the portion of the object using the image.

Example 3—The device in any of Examples 1-2, wherein the first message further indicates a request for medical product information.

Example 4—The device in any of Examples 1-3, wherein the first message further indicates a request for instructions for use, and wherein the medical product data comprises the instructions for use.

Example 5—The device in any of Examples 1-4, wherein the portion of the object comprises at least a product identifier, a label, a code, a quick response (QR) code, a brand name, a model name, a model number, or a regulator identifier.

Example 6—The device in any of Examples 1-5, wherein the medical product data is retrieved from a database.

Example 7—The device in any of Examples 1-6, wherein the processor is further configured to train the product detection model with product detection data that includes an image of the medical product.

Example 8—The device in any of Examples 1-7, wherein the processor is further configured to generate the computer-generated image of the medical product using a computer aided drawing associated with the medical product.

Example 9—The device in any of Examples 1-7, wherein the processor is further configured to generate the computer-generated image by using domain randomization to assist the product detection model in focusing on a feature of the medical product.

Example 10—The device in any of Examples 1-7, wherein the processor is further configured to generate the computer generated image of the medical product by generating a synthetic product image of a medical product using a computer aided drawing for the medical product, wherein the synthetic product image is a photo realistic image; generating a medical environment image, wherein the medical environment image is an image of least one of a hospital room, a medical office, or operating room; and generating the computer generated image of the medical product by imposing the synthetic product image onto the medical environment image.

Example 11—The device in any of Examples 1-7, wherein the processor is further configured to generate the computer generated image of the medical product by generating a synthetic product image of a medical product using a computer aided drawing for the medical product, wherein the synthetic product image is a photo realistic image; generating a medical environment image, wherein the medical environment image is an image of least one of a hospital room, a medical office, or operating room; generating a medical object, wherein the medical object is associated with the medical environment and the medical product; and generating the computer generated image of the medical product by imposing the synthetic product image and the medical object onto the medical environment image.

Example 12—The device in any of Examples 1-11, wherein the confidence level is a first confidence level, wherein the threshold is a first threshold, and wherein the processor is further configured to generate a second confidence level associated with a counterfeit product, wherein the generated second confidence level is based on the portion of the object and a counterfeit detection model that has been trained using counterfeit detection data, and wherein the counterfeit detection data comprises at least an image of a counterfeit product; and identify the object as an authentic product when the confidence level satisfies a second threshold.

Example 13—A method used by a device for providing a medical product assistant to identify a medical product. The method may comprise receiving a first message from a user interface, wherein the first message indicates a request from a user to identify an object; determining a portion of the object using a camera of the device. The method may comprise generating a confidence level associated with a medical product, wherein the generated confidence level indicates a confidence that the object is the medical product, wherein the generated confidence level is based on the portion of the object and a product detection model, and wherein the product detection model has been trained using synthetic product detection data that comprises at least a computer generated image of the medical product. The method may comprise identifying the object as the medical product when the confidence level satisfies a threshold. The method may comprise sending a second message to the user interface when the object is identified, wherein the second message indicates that the object is the medical product, indicates a product identifier for the medical product, and indicates medical product data.

Example 14—The method in any of Example 13, wherein the method further comprises, determining a video stream using the camera, determining an image from the video stream, and determining the portion of the object using the image.

Example 15—The method in any of Examples 13-14, wherein the first message further indicates a request for medical product information.

Example 16—The method in any of Examples 13-15, wherein the first message further indicates a request for instructions for use, and wherein the medical product data comprises the instructions for use.

Example 17—The method in any of Examples 13-16, wherein the portion of the object comprises at least a product identifier, a label, a code, a quick response (QR) code, a brand name, a model name, a model number, or a regulator identifier.

Example 18—The method in any of Examples 13-17, wherein the medical product data is retrieved from a database.

Example 19—The method in any of Examples 13-18, wherein the method further comprises training the product detection module with product detection data that includes an image of the medical product.

Example 20—The method in any of Examples 13-19, wherein the method further comprises generating the computer-generated image of the medical product using a computer-aided drawing associated with the medical product.

Example 21—The method in any of Example 13-19, wherein the method further comprises generating the computer-generated image by using domain randomization to assist the product detection model in focusing on a feature of the medical product.

Example 22—The method in any of Examples 13-19, wherein the method further comprises generating the computer generated image of the medical product by generating a synthetic product image of a medical product using a computer aided drawing for the medical product, wherein the synthetic product image is a photo realistic image; generating a medical environment image, wherein the medical environment image is an image of least one of a hospital room, a medical office, or operating room; and generating the computer generated image of the medical product by imposing the synthetic product image onto the medical environment image.

Example 23—The method in any of Examples 13-19, wherein the method further comprises generating the computer generated image of the medical product by generating a synthetic product image of a medical product using a computer aided drawing for the medical product, wherein the synthetic product image is a photo realistic image; generating a medical environment image, wherein the medical environment image is an image of least one of a hospital room, a medical office, or operating room; generating a medical object, wherein the medical object is associated with the medical environment and the medical product; and generating the computer generated image of the medical product by imposing the synthetic product image and the medical object onto the medical environment image.

Example 24—The method in any of Example 13-23, wherein the confidence level is a first confidence level, wherein the threshold is a first threshold, and wherein the method further comprises generating a second confidence level associated with a counterfeit product, wherein the generated second confidence level is based on the portion of the object and a counterfeit detection model that has been trained using counterfeit detection data, and wherein the counterfeit detection data comprises at least an image of a counterfeit product; and identifying the object as an authentic product when the confidence level satisfies a second threshold.

Example 25—A device for providing a medical product assistant to identify a medical product, the device comprising a processor. The processor may be configured to receive a first message from a user interface, wherein the first message indicates a request from a user to identify an object. The processor may be configured to determine a portion of the object using a camera of the device. The processor may be configured to generate a first confidence level associated with a first medical product, wherein the generated first confidence level indicates a confidence that the object is the first medical product, and wherein the generated first confidence level is based on the portion of the object and a product detection model, and wherein the product detection module has been trained using synthetic product detection data that comprises at least a computer generated image of the first medical product. The processor may be configured to generate a second confidence level associated with a second medical product, wherein the generated second confidence level indicates a confidence that the object is the second medical product. The processor may be configured to identify the object as the first medical product when the first confidence level is greater than the second confidence level, and the first confidence level satisfies a threshold. The processor may be configured to send a second message to the user interface when the object is identified, wherein the second message indicates that the object is the first medical product, indicates a product identifier for the first medical product, and indicates medical product data for the first medical product.

Example 26—The device of Example 25, wherein the product detection model is at least a deep learning model, a convolutional neural network model, or a recurrent neural network model.

Example 27—The device in any of Examples 25-26, wherein the product detection data comprises at least one of an image of the first medical product, information about the first medical product, a 3D model of the first medical product, a computer aided design of the first medical model, a bill of materials for the first medical product, the color of the first medical product, a size of the first medical product, shape of the first medical product, a logo associated with the first medical product, a name of the first medical product, a brand associated with the first medical product, a manufacturer of the first medical product, or instructions for using the first medical product.

Example 28—The device in any of Examples 25-27, wherein the processor is further configured to receive a third message from the user input, wherein the message input indicates a request for the first medical product data.

Example 29—The device in any of Examples 25-28, wherein the medical product data comprises at least an image of the first medical product, instructions for using the first medical product, a list of compatible medical products for use with the first medical product, a list of warnings for the first medical product, or a list of indications for use of the first medical product.

Example 30—The device in any of Examples 25-29, wherein the processor is further configured to send a third message to the user interface when the first confidence level is below the threshold, wherein the third message indicates the object was not identified.

Example 31—The device in any of Examples 25-30, wherein the processor is further configured to determine the portion of the object using the camera of the device by sending a third message to the user interface, wherein the third message comprises an instruction for taking a picture of the portion of the object from an angle; and receiving a fourth message from the user interface, wherein the fourth message comprises an image of the portion of the object taken from the angle.

Example 32—The device in any of Examples 25-30, wherein the processor is further configured to determine the portion of the object using the camera of the device by sending a third message to the user interface, wherein the third message comprises an instruction for taking a picture of the portion of the object with better lighting; and receiving a fourth message from the user interface, wherein the fourth message comprises an image of the object taken with better lighting.

Example 33—The device in any of Examples 25-32, wherein the processor is further configured to receive the first message from the user interface by determining that the first message includes a voice recording; and determining the request from the user to identify the object from the voice recording using a voice recognition module.

Example 34—A method used by a device for providing a medical product assistant to identify a medical product. The method may comprise receiving a first message from a user interface, wherein the first message indicates a request from a user to identify an object. The method may comprise determining a portion of the object using a camera of the device. The method may comprise generating a first confidence level associated with a first medical product, wherein the generated first confidence level indicates a confidence that the object is the first medical product, and wherein the generated first confidence level is based on the portion of the object and a product detection model, and wherein the product detection module has been trained using synthetic product detection data that comprises at least a computer generated image of the first medical product. The method may comprise generating a second confidence level associated with a second medical product, wherein the generated second confidence level indicates a confidence that the object is the second medical product. The method may comprise identifying the object as the first medical product when the first confidence level is greater than the second confidence level, and the first confidence level satisfies a threshold. The method may comprise sending a second message to the user interface when the object is identified, wherein the second message indicates that the object is the first medical product, indicates a product identifier for the first medical product, and indicates medical product data for the first medical product.

Example 35—The method of Example 34, wherein the product detection model is at least a deep learning model, a convolutional neural network model, or a recurrent neural network model.

Example 36—The method in any of Examples 34-35, wherein the product detection data comprises at least one of an image of the first medical product, information about the first medical product, a 3D model of the first medical product, a computer aided design of the first medical model, a bill of materials for the first medical product, the color of the first medical product, a size of the first medical product, shape of the first medical product, a logo associated with the first medical product, a name of the first medical product, a brand associated with the first medical product, a manufacturer of the first medical product, or instructions for using the first medical product.

Example 37—The method in any of Example 34-36, wherein the method further comprises receiving a third message from the user input, wherein the message input indicates a request for the first medical product data.

Example 38—The method in any of Examples 34-37, wherein the medical product data comprises at least an image of the first medical product, instructions for using the first medical product, a list of compatible medical products for use with the first medical product, a list of warnings for the first medical product, or a list of indications for use of the first medical product.

Example 39—The method in any of Examples 34-38, wherein the method further comprises sending a third message to the user interface when the first confidence level is below the threshold, wherein the third message indicates the object was not identified.

Example 40—The method in any of Examples 34-39, wherein the method further comprises determining the portion of the object using the camera of the device by sending a third message to the user interface, wherein the third message comprises an instruction for taking a picture of the portion of the object from an angle; and receiving a fourth message from the user interface, wherein the fourth message comprises an image of the portion of the object taken from the angle.

Example 41—The method in any of Examples 34-38, wherein the method further comprises determining the portion of the object using the camera of the device by sending a third message to the user interface, wherein the third message comprises an instruction for taking a picture of the portion of the object with better lighting; and receiving a fourth message from the user interface, wherein the fourth message comprises an image of the object taken with better lighting.

Example 42—The method of Example 34-41, wherein the method further comprises receiving the first message from the user interface by determining that the first message includes a voice recording; and determining the request from the user to identify the object from the voice recording using a voice recognition module.

Example 43—A device for providing a medical product assistant to identify a medical product, the device comprising a processor. The processor may be configured to receive a first message from a user interface, wherein the first message indicates a request from a user to identify an object. The processor may be configured to determine a portion of the object using a camera of the device. The processor may be configured to generate a confidence level associated with a medical product package, wherein the generated confidence level indicates a confidence that the object is the medical product package, wherein the generated confidence level is based on the portion of the object and a package detection model, and wherein the package detection module has been trained using synthetic package detection data that comprises at least a computer generated image of a package for a medical product. The processor may be configured to identify the object as the medical product when the confidence level satisfies a threshold. The processor may be configured to send a second message to the user interface when the object is identified, wherein the second message indicates that the object is the medical product, indicates a product identifier for the medical product, and indicates medical product data.

Example 44—The device of Example 43, wherein the portion of the object comprises at least a product identifier, a label, a code, a quick response (QR) code, a brand name, a model name, a model number, or a regulator identifier.

Example 45—The device in any of Examples 43-44, wherein the processor is further configured to generate the computer-generated image of the medical product package using a computer-aided drawing associated with the medical product.

Example 46—The device in any of Example 43-44, wherein the processor is further configured to generate the computer-generated image by using domain randomization to assist the product detection model in focusing on a feature of the medical product.

Example 47—The device in any of Examples 43-44, wherein the processor is further configured to generate the computer generated image of the medical product package by generating a synthetic product image of a medical product package using a computer aided drawing for the medical product, wherein the synthetic product image is a photo realistic image; generating a medical environment image, wherein the medical environment image is an image of at least one of a hospital room, a medical office, or operating room; and generating the computer generated image of the medical product package by imposing the synthetic product image onto the medical environment image.

Example 48—The device in any of Examples 43-44, wherein the processor is further configured to generate the computer generated image of the medical product by generating a synthetic product image of a medical product package using a computer aided drawing for the medical product, wherein the synthetic product image is a photo realistic image generating a medical environment image, wherein the medical environment image is an image of least one of a hospital room, a medical office, or operating room; generating a medical object, wherein the medical object is associated with the medical environment and the medical product; and generating the computer generated image of the medical product package by imposing the synthetic product image and the medical object onto the medical environment image.

Example 49—The device in any of Examples 43-48, wherein the confidence level is a first confidence level, wherein the threshold is a first threshold, and wherein the processor is further configured to generate a second confidence level associated with a counterfeit product, wherein the generated second confidence level is based on the portion of the object and a counterfeit detection model that has been trained using counterfeit detection data, and wherein the counterfeit detection data comprises at least an image of a counterfeit product package; and identify the object as an authentic product when the confidence level satisfies a second threshold.

Example 50—A method used by a device for providing a medical product assistant to identify a medical product. The method may comprise receiving a first message from a user interface, wherein the first message indicates a request from a user to identify an object. The method may comprise determining a portion of the object using a camera of the device. The method may comprise generating a confidence level associated with a medical product package, wherein the generated confidence level indicates a confidence that the object is the medical product package, and wherein the generated confidence level is based on the portion of the object and a package detection model, wherein the package detection module has been trained using synthetic package detection data that comprises at least a computer generated image of a package for a medical product. The method may comprise identifying the object as the medical product when the confidence level satisfies a threshold. The method may comprise sending a second message to the user interface when the object is identified, wherein the second message indicates that the object is the medical product, indicates a product identifier for the medical product, and indicates medical product data.

Example 51—The method of Example 50, wherein the portion of the object comprises at least a product identifier, a label, a code, a quick response (QR) code, a brand name, a model name, a model number, or a regulator identifier.

Example 52—The method in any of Examples 50-51, wherein method further comprises generating the computer-generated image of the medical product package using a computer aided drawing associated with the medical product.

Example 53—The method in any of Examples 50-51, wherein the method further comprises generating the computer-generated image by using domain randomization to assist the product detection model in focusing on a feature of the medical product.

Example 54—The method in any of Examples 50-51, wherein method further comprises generating the computer generated image of the medical product package by generating a synthetic product image of a medical product package using a computer aided drawing for the medical product, wherein the synthetic product image is a photo realistic image; generating a medical environment image, wherein the medical environment image is an image of least one of a hospital room, a medical office, or operating room; and generating the computer generated image of the medical product package by imposing the synthetic product image onto the medical environment image.

Example 55—The method in any of Examples 50-51, wherein the method for the comprises generating the computer generated image of the medical product by generating a synthetic product image of a medical product package using a computer aided drawing for the medical product, wherein the synthetic product image is a photo realistic image; generating a medical environment image, wherein the medical environment image is an image of least one of a hospital room, a medical office, or operating room; generating a medical object, wherein the medical object is associated with the medical environment and the medical product; and generating the computer generated image of the medical product package by imposing the synthetic product image and the medical object onto the medical environment image.

Example 56—The method of Examples 50-55, wherein the confidence level is a first confidence level, wherein the threshold is a first threshold, and wherein the method further comprises generating a second confidence level associated with a counterfeit product, wherein the generated second confidence level is based on the portion of the object and a counterfeit detection model that has been trained using counterfeit detection data, and wherein the counterfeit detection data comprises at least an image of a counterfeit product package; and identifying the object as an authentic product when the confidence level satisfies a second threshold.

This application may refer to “determining” various pieces of information. Determining the information can include one or more of, for example, estimating the information, calculating the information, predicting the information, or retrieving the information from memory.

Additionally, this application may refer to “receiving” various pieces of information. Receiving is, as with “accessing,” intended to be a broad term. Receiving the information can include one or more of, for example, accessing the information, or retrieving the information (for example, from memory). Further, “receiving” is typically involved, in one way or another, during operations such as, for example, storing the information, processing the information, transmitting the information, moving the information, copying the information, erasing the information, calculating the information, determining the information, predicting the information, or estimating the information.

It is to be appreciated that the use of any of the following “/,” “and/or,” and “at least one of,” for example, in the cases of “A/B,” “A and/or B” and “at least one of A and B,” is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as is clear to one of ordinary skill in this and related arts, for as many items as are listed.

We describe a number of examples. Features of these examples can be provided alone or in any combination, across various claim categories and types. Further, embodiments can include one or more of the following features, devices, or aspects, alone or in any combination, across various claim categories and types.

As used herein, term “message” may encompass a variety of forms and formats, which may be transmitted, received, or otherwise manipulated via a series of coded information or signals. A signal may include, a means or mechanism to convey, deliver, or disseminate a message or set of information through electromagnetic waves, optical means, or any other transmission medium.

The processes and examples described above may be implemented in a computer program, software, and/or firmware incorporated in a computer-readable medium for execution by a computer and/or processor. Examples of computer-readable media include, but are not limited to, electronic signals (transmitted over wired and/or wireless connections) and/or computer-readable storage media. Examples of computer-readable storage media include, but are not limited to, a read only memory (ROM), a random access memory (RAM), a register, cache memory, semiconductor memory devices, magnetic media such as, but not limited to, internal hard disks and removable disks, magneto-optical media, and/or optical media such as compact disc (CD)-ROM disks, and/or digital versatile disks (DVDs). A processor in association with software may be used to implement a radio frequency transceiver for use in a WTRU, terminal, base station, RNC, and/or any host computer.

1. A device for providing a medical product assistant, the device comprising:
a processor, wherein the processor is configured to:
receive a first message from a user interface, wherein the first message indicates a request from a user to identify an object;
determine a portion of the object using a camera of the device;
generate a confidence level associated with a medical product, wherein the generated confidence level indicates a confidence that the object is the medical product, wherein the generated confidence level is based on the portion of the object and a product detection model, and wherein the product detection model has been trained using synthetic product detection data that comprises at least a computer-generated image of the medical product;
identify the object as the medical product when the confidence level satisfies a threshold; and
send a second message to the user interface when the object is identified, wherein the second message indicates that the object is the medical product, indicates a product identifier for the medical product, and indicates medical product data.

2. The device of claim 1, wherein the processor is further configured to determine the portion of the object using the camera of the device by:
determining a video stream using the camera;
determining an image from the video stream; and
determining the portion of the object using the image.

3. The device of claim 1, wherein the first message further indicates a request for medical product information.

4. The device of claim 1, wherein the first message further indicates a request for instructions for use, and wherein the medical product data comprises the instructions for use.

5. The device of claim 1, wherein the portion of the object comprises at least a product identifier, a label, a code, a quick response (QR) code, a brand name, a model name, a model number, or a regulator identifier.

6. The device of claim 1, wherein the medical product data is retrieved from a database.

7. The device of claim 1, wherein the processor is further configured to train the product detection model with product detection data that includes an image of the medical product.

8. The device of claim 1, wherein the processor is further configured to generate the computer-generated image of the medical product using a computer aided drawing associated with the medical product.

9. The device of claim 1, wherein the processor is further configured to generate the computer-generated image by using domain randomization to assist the product detection model in focusing on a feature of the medical product.

10. The device of claim 1, wherein the processor is further configured to generate the computer-generated image of the medical product by:
generating a synthetic product image of a medical product using a computer aided drawing for the medical product, wherein the synthetic product image is a photo realistic image;
generating a medical environment image, wherein the medical environment image is an image of least one of a hospital room, a medical office, or operating room; and
generating the computer-generated image of the medical product by imposing the synthetic product image onto the medical environment image.

11. The device of claim 1, wherein the processor is further configured to generate the computer-generated image of the medical product by:
generating a synthetic product image of a medical product using a computer aided drawing for the medical product, wherein the synthetic product image is a photo realistic image;
generating a medical environment image, wherein the medical environment image is an image of least one of a hospital room, a medical office, or operating room;
generating a medical object, wherein the medical object is associated with the medical product and at least one of the hospital room, the medical office, or the operating room; and
generating the computer-generated image of the medical product by imposing the synthetic product image and the medical object onto the medical environment image.

12. The device of claim 1, wherein the confidence level is a first confidence level, wherein the threshold is a first threshold, and wherein the processor is further configured to:
generate a second confidence level associated with a counterfeit product, wherein the generated second confidence level is based on the portion of the object and a counterfeit detection model that has been trained using counterfeit detection data, and wherein the counterfeit detection data comprises at least an image of a counterfeit product; and
identify the object as an authentic product when the confidence level satisfies a second threshold.

13. A method used by a device for providing a medical product assistant, the method comprising:
receiving a first message from a user interface, wherein the first message indicates a request from a user to identify an object;
determining a portion of the object using a camera of the device;
generating a confidence level associated with a medical product, wherein the generated confidence level indicates a confidence that the object is the medical product, wherein the generated confidence level is based on the portion of the object and a product detection model, and wherein the product detection model has been trained using synthetic product detection data that comprises at least a computer-generated image of the medical product;
identifying the object as the medical product when the confidence level satisfies a threshold; and
sending a second message to the user interface when the object is identified, wherein the second message indicates that the object is the medical product, indicates a product identifier for the medical product, and indicates medical product data.

14. The method of claim 13, wherein the method further comprises:
determining a video stream using the camera;
determining an image from the video stream; and
determining the portion of the object using the image.

15. The method of claim 13, wherein the first message further indicates a request for medical product information.

16. The method of claim 13, wherein the first message further indicates a request for instructions for use, and wherein the medical product data comprises the instructions for use.

17. The method of claim 13, wherein the portion of the object comprises at least a product identifier, a label, a code, a quick response (QR) code, a brand name, a model name, a model number, or a regulator identifier.

18. The method of claim 13, wherein the medical product data is retrieved from a database.

19. The method of claim 13, wherein the method further comprises training the product detection model with product detection data that includes an image of the medical product.

20. The method of claim 13, wherein the method further comprises generating the computer-generated image of the medical product using a computer aided drawing associated with the medical product.
